(19) United States
(12) Patent Application Publication
(10) Pub. No.: US 2016/0256097 A1
(43) Pub. Date: Sep. 8, 2016

(54) POLLEN SAMPLING AND RETRIEVAL TRIGGERED BY A USER'S ALLERGIC REACTIONS

(71) Applicant: Scanit Technologies, Inc., Milpitas, CA (US)

(72) Inventors: Pedro Manautou, Milpitas, CA (US); Joel Kent, Fremont, CA (US)

(21) Appl. No.: 15/061,883

(22) Filed: Mar. 4, 2016

Related U.S. Application Data
(60) Provisional application No. 62/129,571, filed on Mar. 6, 2015.
(60) Provisional application No. 62/188,606, filed on Jul. 3, 2015.

(51) Int. Cl. A61B 5/00; G01N 15/06; G01N 1/22

(52) U.S. Cl. CPC A61B 5/411; G01N 1/2273; G01N 15/0606

(57) ABSTRACT

Information from a sensor that is local to a user is received. The information may indicate that the user has experienced a physiological event. The information is analyzed to determine whether the physiological event should be classified as an allergic reaction. If the physiological event should be classified as an allergic reaction, an action is taken such as particles currently present in the environment local to the user are collected.

### CROSS-REFERENCE TO RELATED APPLICATIONS

This patent application claims the benefit of U.S. provisional patent applications 62/129,571, filed Mar. 6, 2015; and 62/188,606, filed Jul. 3, 2015, all of which are incorporated by reference along with all other references cited in this application.

### BACKGROUND

The present invention relates to the field of health monitoring, and more specifically, to personal pollen sampling, data logging and archiving devices and associated devices that monitor allergic reactions of users.

Millions upon millions of people worldwide suffer allergies. An allergy is the response of the body's immune system to a substance to which the body has become hypersensitive. The immune system mistakes an otherwise harmless substance as being harmful and over-reacts. Allergies can range from mild discomfort to life-threatening.

Pollen is one example of a substance to which many people are allergic. Other types of substances include fur, particular foods, dust, or mold, among many others. For some people, these substances do not present a problem. Other people, however, may suffer an allergy or have an allergic reaction. Hay fever is an allergy that can be caused by pollen. Hay fever symptoms include a runny nose, nasal congestion, watery eyes, itchy eyes, sneezing, coughing, itchy nose, itchy roof of mouth, itchy throat, sinus pressure, facial pain, swollen eyes, decreased sense of smell, or a decreased sense of taste.

Diagnosing and treating an allergy, however, is very difficult because of the many different types of substances that people encounter throughout their daily lives. For example, there are many different types of pollen such as grass pollen (e.g., ryegrass or timothy), tree pollen (e.g., birch, alder, cedar, hazelnut, willow, plane, olive, or hornbeam), weed pollen (e.g., ragweed, nettle, mugwort, goosefoot, or sorrel), and many others. Different people react differently to different types of pollen. A person may be affected by one type of pollen and unaffected by another type of pollen. During consultation at a doctor's office, it may be difficult to determine the specific allergen(s) involved because a pollen sample from the environment where the user suffers allergic reactions may not be available.

Furthermore, a complicating factor is that not every airborne particle in the user's environment is an allergenic pollen grain. For example, the user's environment may well be subjected to a number of non-allergenic pollens as well as allergenic pollen(s). This leads to two problems.

First, when several types of potentially allergenic pollen are collected from the user's environment, it may not be clear which one(s) is causing problems. Secondly, the presence of non-allergenic pollen increases the burden of analysis of pollen samples, whether done manually via microscope or whether done using automated methods.

Both of these problems would be reduced if pollen samples could be selectively collected when allergenic pollen(s) is known to be present. Such selective sampling would reduce the number of particles that need to be analyzed. Furthermore, particularly if control samples could also be collected when the user does not have an allergic reaction, such selective sampling would provide better information for determining which collected pollen types are allergenic for the user. A more precise knowledge of the specific allergen(s) involved can benefit such sufferers or “users” by pointing the way to appropriate therapies, coping strategies, or both.

### BRIEF SUMMARY OF THE INVENTION

In a specific embodiment, information from a sensor that is local to a user is received. The information may indicate that the user has experienced a physiological event. The information is analyzed to determine whether the physiological event should be classified as an allergic reaction. If the physiological event should be classified as an allergic reaction, particles currently present in the environment local to the user are collected.

In another specific embodiment, a method includes periodically collecting over a rolling time period candidate priming pollens in an environment local to a user, receiving from a sensor local to the user information indicating that the user has experienced a physiological event, analyzing the information to determine whether the physiological event should be classified as an allergic reaction, based on the analysis, determining that the physiological event should be classified as the allergic reaction, upon the determination, collecting pollens currently present in the environment local to the user, identifying the current pollens and the candidate priming pollens, scanning a table comprising a listing of pollens, and a listing of priming pollens corresponding to the listing of pollens to find a specific pollen among the listing of pollens that is present in the current pollens, and a specific priming pollen among the listing of priming pollens, corresponding to the specific pollen, that is present in the candidate priming pollens; and generating a notification comprising an identification of the specific pollen, and an identification of the specific priming pollen that corresponds to the specific pollen.

In another specific embodiment, a method includes collecting over a rolling time period airborne particles in an environment local to a user, receiving from a sensor associated with the user information indicating that the user has experienced a physiological event, analyzing the information to determine whether the physiological event should be classified as an allergic reaction, if the physiological event should be classified as an allergic reaction, correlating first particles collected during a first portion of the rolling time period with second particles collected during a second portion of the rolling time period to identify an aggravating allergen among the first particles, and a priming allergen, corresponding to the aggravating allergen, among the second particles, where a duration of the first portion of the rolling time period is less than a duration of the second portion of the rolling time period, and where the first portion of the rolling time period is closer to a time of the physiological event than the second portion of the rolling time period.

Selective pollen sampling may be enabled with a personal pollen collection system including a pollen sampler and an allergic reaction monitor in which pollen sampling is triggered when the monitor detects a user's allergic reaction. The sampler may, for example, collect sampled pollen on an adhesive layer on tape or a glass slide for later inspection. The monitor may, for example, include a microphone system listening for sounds characteristic of sneezing or coughing.

Optionally, the personal pollen collection system also includes means to automatically identify the type(s) of pollen sampled. Automated pollen recognition may be based on image processing, non-image optical properties such as scattering and fluorescence, or combinations of these. In a specific embodiment, a pollen collection system includes an adhesive coated tape upon which pollen samples are collected. The tape including the physical pollen samples may be archived for later retrieval. Data associated with the analysis of the physical pollen samples may be logged. The archiving of physical pollen samples, past data analysis, or both can be later retrieved to provide insights on what may have primed the user allergic reactions.

Optionally the system may be in communication with a network receiving information from the personal pollen collection system, providing information to the personal pollen collection system, or both. The network may, for example, be informed of the time, location, environmental conditions (temperature, humidity, etc.) and a sample identifier number for each pollen sample collected. The network may, for example, provide the personal pollen collection system with relevant contextual information such as the types of allergenic pollen likely to be present given the season, weather conditions, pollen forecasts, and the location of the personal pollen collection system.

Optionally, the pollen collection system may well be a set of pollen collection systems in which the pollen collection system or systems closest to the user collect samples when triggered by the user's allergic reaction monitor. For example, a user might place a collection system in their bedroom, living room, kitchen and automobile. In another example, an assisted-living community could place collection systems in all buildings and outdoor locations frequented by aging and forgetful residents. In yet another example, pollen collection systems may be incorporated into personal or companion robots that generally follow the humans they serve.

In a specific embodiment, a personal pollen collection system includes a pollen sampler and an allergic reaction monitor. Pollen sampling is triggered when the monitor detects a user's allergic reaction. The sampler may collect sampled pollen on an adhesive layer on tape or a glass slide for later inspection. The monitor may include a microphone system listening for sounds characteristic of user sneezing or coughing. There may be one sampler associated with a user associated with an allergic reaction monitor. Alternatively or additionally, the monitor may communicate (e.g., via Bluetooth or a cloud service) with a larger set of monitors in order to trigger pollen sampling by the monitor(s) in the closest proximity

It should be appreciated that aspects and principles of the system may be applied to other allergens and pathogens besides pollen.

Other objects, features, and advantages will become apparent upon consideration of the following detailed description and the accompanying drawings, in which like reference designations represent like features throughout the figures.

### BRIEF DESCRIPTION OF THE FIGURES

FIG. 1 shows a block diagram of a system for pollen sampling and retrieval triggered by a user's allergic reaction.

FIG. 2A shows a block diagram of an analysis server.

FIG. 2B shows a block diagram of a local particle collection device.

FIG. 3 shows a block diagram of a mobile communications device having an allergen analysis mobile application program.

FIG. 4 shows a block diagram of a wearable computer having an allergen analysis mobile application program.

FIG. 5 shows options for wearable allergic reaction monitors.

FIG. 6 shows a floor plan of a house illustrating an example deployment of the system.

FIG. 7 shows an overall flow diagram of a process for sampling ambient air according to a specific embodiment.

FIG. 8 shows an overall flow diagram of another process for sampling ambient air according to a specific embodiment.

FIG. 9 shows a flow of a process for deploying and using the system.

FIG. 10 shows a flow of a process for generating an allergic reaction signature.

FIG. 11 shows a flow of a process for prompting the user to verify an allergic reaction.

FIG. 12 shows a flow of a process for identifying an aggravating allergen.

FIG. 13 shows a timeline of events associated with an allergic reaction.

FIG. 14 shows a flow of a process for identifying aggravating and priming allergens.

FIG. 15 shows another time of events associated with an allergic reaction.

FIG. 16 shows a side view of a particle collection device according to a specific embodiment.

FIG. 17 shows another side view of the particle collection device.

FIG. 18 shows a plan view of the particle collection device.

FIG. 19 shows another plan view of the particle collection device.

FIG. 20 shows a block diagram of a client-server system and network in which an embodiment of the system may be implemented.

FIG. 21 shows a system block diagram of a client computer system.

FIG. 22 shows an exterior view of an alternate particle collection device according to another specific embodiment.

FIG. 23A shows an isometric view of a particle media cartridge that may be used with the alternate particle collection device.

FIG. 23B shows a plan view of a cross section of the particle media cartridge.

FIG. 23C shows a plan view of a cross section of the particle media cartridge including media.

FIG. 24 shows a plan-view of the alternate particle collection device including motors.

FIG. 25 shows a vertical cross-section of the alternate particle collection device illustrating air flow.

FIG. 26 shows some detail of the alternate particle collection device with optics and particle media cartridge.

FIG. 27A shows a vertical cross-section of the alternate particle collection device including electronics.

FIG. 27B shows an example of a kit including particle collection cartridges.

FIG. 28 show an allergic reaction timing diagram.

FIG. 29 shows a line chart that plots allergic reaction severity against time.

### DETAILED DESCRIPTION

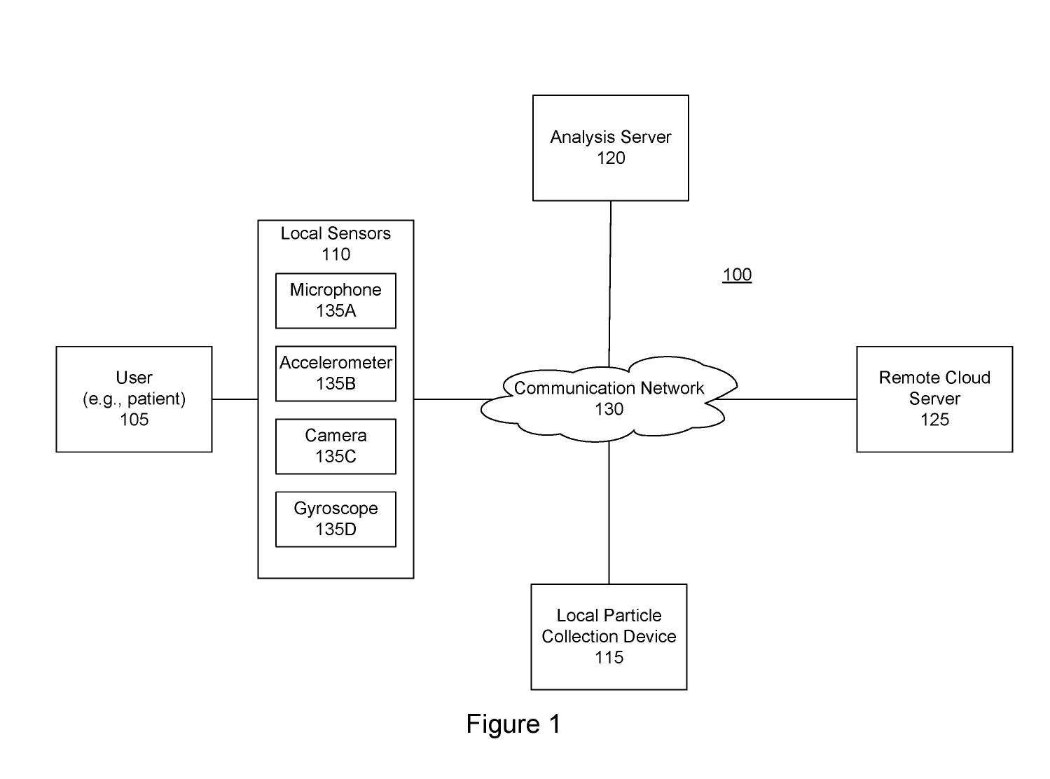

FIG. 1 shows a simplified block diagram of a system 100 for monitoring an environment of a user 105. The user may be referred to as a patient. The system collects and analyses airborne or floating particles. In a specific embodiment, the system correlates, associates, or links collecting with an allergic reaction suffered by the user. This helps to narrow and identify the types of allergens that may contribute to the allergic reaction. With this information, a treatment plan can then be developed to reduce or eliminate future allergic reactions. The collected particles may include pollen, mold spores, animal dander (e.g., tiny flecks of skin shed by cats, dogs, and birds), or any other allergenic particles that may be present in the user's personal or local environment. This system includes one or more sensors 110, a particle or pollen collection device or machine 115, an analysis server 120, and a remote cloud server 125, each of which are interconnected by a network 130.

The sensor may be incorporated into a device that may be referred to as an allergic reaction monitoring device. The allergic reaction monitoring device may be implemented as a mobile device of the user such as a smartphone or smartwatch. The mobile device executes a mobile application program or app that includes executable or computer-readable code that embodies a technique or algorithm as described in this application. For example, the mobile application may analyze a signal generated from the sensor to determine whether or not the user suffered an allergic reaction. In another specific embodiment, the allergic reaction monitoring device is a physical device that is separate from the user's mobile communication device. In this specific embodiment, the allergic reaction monitoring device may include, in addition to a sensor, components such as a battery, power cord, power converter (e.g., AC/DC converter), antenna, communication protocol, network interface, memory, storage, processor, and so forth. The allergic reaction monitoring device may be associated with an Internet Protocol (IP) address so that it can communicate and exchange information with other components of the system over a network such as the Internet.

In a specific embodiment, the monitoring device is local to the user and monitors the user for particular changes or events. In a specific embodiment, the device is a non-invasive device that may be attached, associated, proximate to, or near the user. Non-invasive techniques as compared to invasive techniques have less risk of infection because there is no cutting or puncturing of the user's body; foreign objects are not introduced or placed inside the user's body. Further, non-invasive techniques are generally less expensive than invasive techniques. In another specific embodiment, however, there can be an invasive sensor in which the sensor is implanted inside or inserted into the user's body.

The sensor can be a microphone 135A, accelerometer 135B, camera 135C, or gyroscope 135D. A microphone converts sound to an electrical signal and can be used to detect sounds associated with a potential allergic reaction (e.g., coughing or sneezing). The accelerometer measures acceleration and can be used to detect user activity or motions associated with the potential allergic reaction. A gyroscope senses orientation can likewise detect user motions associated with the potential allergic reaction. While not shown, sensor systems that detect the direction of Earth's gravity, the direction of Earth's magnetic field, or both may also be used to detect user motions. A camera is an optical instrument that captures images and can be used to capture images of the user's face during a potential allergic reaction.

There can be multiple or any number of allergic reaction monitoring devices or sensors such as 2, 3, 4, 5, 6, 7, 8, 9, 10, 11, 12, 13, 14, 15, or more than 15 sensors. An implementation may include a combination of sensor types. For example, there can be accelerometers, gyroscopes, and microphones. There can be a microphone clipped to the user's shirt, other microphones may be placed in the user's house, office, or car, an accelerometer and gyroscope may be strapped to the user's wrist, a camera may be built-in to the user's eye glasses, or combinations of these.

The allergic reaction monitoring device with sensor is responsible for detecting physiological events that the user may experience and transmitting information associated with the event to the analysis server for analysis. Some examples of physiological events that the user may experience when encountering certain types of airborne particles, such as certain types of pollen, include coughing, sneezing, wheezing, sniffling, facial contortions that may be associated with pain, tears or watery eyes, changes in eye color, red eyes, or inflamed eyes (e.g., changes in the conjunctiva), swollen skin under the eyes, blue-colored skin under the eyes, a reflexive hand movement or gesture to cover the mouth when sneezing or coughing, changes in voice such as due to nasal congestion or an irritated throat, or combinations of these.

The allergic reaction monitoring device may, as a function of time, simply determine whether or not a user is suffering from an allergic reaction. However, in many cases it is advantageous to provide a greater degree of information about the user's allergic reaction. For example, the strength or degree of allergic reaction (as a function of time) may be provided. The allergic reaction monitoring device may make a determination of the type of allergic reaction, e.g., sneezing vs. deep coughing vs. inflamed eyes, etc. A distinction may also be made between acute versus chronic allergic reactions. Such detail on the nature of the user's allergic reaction may influence details on how pollen sampling is triggered and executed.

As an example of how the nature of a user's allergic reaction may influence details on how pollen sampling is triggered and executed, it is of interest to consider the case that the allergic reaction monitor collects sufficient information to distinguish between “early phase” symptoms related to the immune systems release of histamine and leukotrienes and other small molecular weight molecules triggered by IgE anti-body recognition of an antigen, and “late phase” symptoms where additional immune cells arrive at the tissue exposed to the antigen (e.g. in the nose). For the purposes of identifying offending allergenic particles, early phase symptoms are much more useful as their timing is much closer to the timing of exposure. While late phase symptoms may be at least as important as early phase symptoms from the perspective of user suffering, late phase symptoms are less useful for the purpose of identifying offending allergens via correlations between timing of exposure and timing of symptoms. In some embodiments, the onset of early phase symptoms triggers additional particle collection beyond a routine particle collection rate for control purposes while late phase symptoms do not.

In a specific embodiment, the system grades or assigns to each allergic reaction a severity rating. For example, a severity rating of 1 may be assigned to very minor or very mild allergic reactions. A severity rating of 3 may be assigned to moderate allergic reactions. A severity rating of 5 may be assigned to very severe allergic reactions. The system may evaluate any number of factors in determining the severity of an allergic reaction. Such factors may include, for example, a duration of the allergic reaction (e.g., how many times did the user the sneeze?, how long did the user cough?), a sound intensity of the allergic reaction (e.g., how loud or how many decibels was the cough?), other factors, or combinations of these. For example, an allergic reaction that involved a series of five consecutive sneezes may be assigned a higher severity rating than allergic reaction that involved a series of three consecutive sneezes.

In a specific embodiment, the system may prompt the user to grade the severity of their allergic reaction. For example, upon determining that a user has suffered an allergic reaction, the system may display on an electronic screen of a smartphone device of the user the message, “Please input a severity rating for the allergic reaction you just had.” The system receives the user inputted value or severity rating and associates the allergic reaction with the received severity rating. A severity rating may be based on a numerical value (e.g., 1, 2, 3, 4, 5, 6, 7, 8, 9, or 10), letter value (e.g., A, B, C, D, or E), or any other type of value.

The system can further log or tag each allergic reaction with a timestamp indicating the time and date that the allergic reaction occurred. Table A below shows an example of data that may be logged and stored by the system.

TABLE A

| Severity | Time and Date |
|---|---|
| 2 | Jan. 2, 2016 8:35 AM (PST) |
| 3 | Jan. 4, 2016 9:27 AM (PST) |
| 2 | Jan. 5, 2016 1:44 PM (PST) |
| 5 | Jan. 5, 2016 12:19 PM (PST) |
| 4 | Jan. 8, 2016 10:11 AM (PST) |
| . . . | . . . |

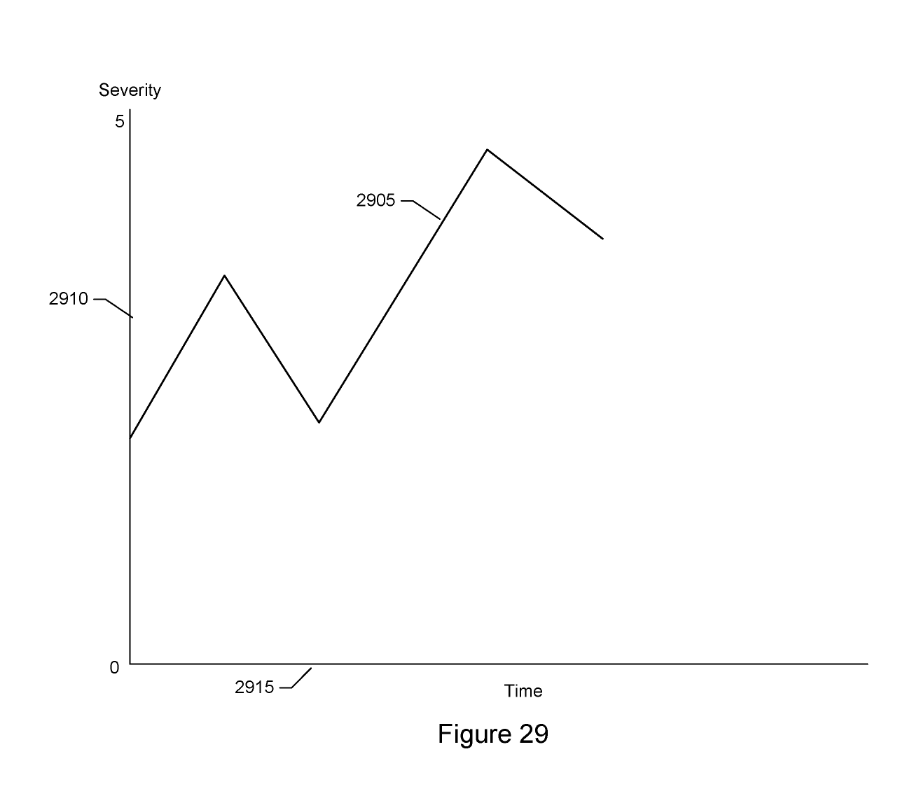

Table A above includes columns or fields labeled “severity” and “time and date.” The “severity” column stores a severity rating of an allergic reaction. The “time and date” column stores a time and date of the allergic reaction. A reporting module of the system may access the log to generate a report that can include a plot, graph, or chart of the logged data. For example, FIG. 29 shows an example of a line chart 2905 that may be generated and displayed by the system (e.g., displayed on an electronic screen). A y-axis 2910 of the chart indicates the severity of the allergic reaction. An x-axis 2915 of the chart indicates chronological time.

In the chart the data shown in table A is displayed as a set of data points, each data point including first and second variables. The first variable determines a position of a point with respect to the x- or horizontal axis. The second variable determines a position of the point with respect to the y- or vertical axis. The points are then joined by a line. In the example shown in FIG. 29, the first variable includes the timestamp value of an allergic reaction. The second variable includes the severity rating of the allergic reaction. The line chart can be used by the user, the user's doctor, or both to visualize trends in the user's history of allergic reactions over a period of time.

In another specific embodiment, the system can tag each allergic reaction with a geotag that indicates the geographical location of an allergic reaction. A geotag may include latitude and longitude, altitude, a place name (e.g., home, office, San Jose Municipal Rose Garden, University of California (UC) Santa Cruz Arboretum, Brooklyn Botanic Garden, or Buehler Vineyards), or combinations of these. Table B below shows an example of the allergic reactions from table A being further tagged with geographical identification metadata.

TABLE B

| Severity | Time and Date | Location |
|---|---|---|
| 2 | Jan. 2, 2016 8:35 AM (PST) | Home |
| 3 | Jan. 4, 2016 9:27 AM (PST) | Office |
| 2 | Jan. 5, 2016 1:44 PM (PST) | Home |
| 5 | Jan. 5, 2016 12:19 PM (PST) | UC Santa Cruz Arboretum |
| 4 | Jan. 8, 2016 10:11 AM (PST) | Buehler Vineyards |
| . . . | . . . | . . . |

The report module of the system can access the data shown in table B above to create graphical representations of the data. For example, the system may generate a bar chart that compares the severity of the allergic reaction with the corresponding location. In this example, there can be a vertical axis that lists the locations, a horizontal axis that shows increasing severity, and a set of bars extending from the vertical axis, where a position of the bar along the vertical axis corresponds to a location of an allergic reaction, and a length of a bar indicates a severity of the allergic reaction. The bar and other charts generated by the system help the user (and the user's doctor) to easily visualize the conditions under which allergic reactions occur. This information can be used to help develop a customized treatment plan. The treatment plan may include an identification of allergy medications, antihistamines, dosage requirements, nasal washes, dust masks, certain places to avoid, when to avoid certain places, and so forth.

In another specific embodiment, the system can tag each allergic reaction with a type value that indicates the symptoms that the user suffered (e.g., sneezing, deep coughing, or inflamed eyes). The system includes logic to identify different types of allergic reactions. In a specific embodiment, the system stores reference or signature data associated with a sneeze, cough, and inflamed eyes. Reference data for a sneeze may include acoustic data characteristics, motion data characteristics, or both that are indicative of a sneeze. Reference data for a cough may include acoustic data characteristics, motion data characteristics, or both that are indicative of a cough. Reference data for inflamed eyes may include images of inflamed eyes.

The system receives, through any number of sensors local to the user, audio data, motion data, image data, or combinations of these. The system can compare the received data against the reference data to determine whether the user suffered an allergic reaction, the type of allergic reaction suffered, or both. In another specific embodiment, the system may prompt the user to input the type of allergic reaction that was suffered.

Table C below shows an example of the allergic reactions from table B being further tagged with the allergic reaction type.

TABLE C

| Severity | Time and Date | Location | Type |
|---|---|---|---|
| 2 | Jan. 2, 2016 8:35 | Home | Sneezing |
| | AM (PST) | | |
| 3 | Jan. 4, 2016 9:27 | Office | Sneezing |
| | AM (PST) | | |
| 2 | Jan. 5, 2016 1:44 PM | Home | Inflamed eyes |
| | (PST) | | |
| 5 | Jan. 5, 2016 12:19 | UC Santa Cruz | Deep coughing |
| | PM (PST) | Arboretum | |
| 4 | Jan. 8, 2016 10:11 | Buehler Vineyards | Deep coughing |
| | AM (PST) | | |
| . . . | . . . | . . . | . . . |

The report module of the system can likewise access the data shown in table C above to create graphical representations of the data. A report or chart can be based on any number of data categories, data sets, or variables (e.g., severity and time, or severity and location). The system can generate any number of different chart types such as bar charts, line charts, area charts, pie charts, scatter plots, or bubble charts—just to name a few examples. The charts may be static charts or animated motion charts.

The analysis server is responsible for analyzing the information detected by the one or more allergic reaction monitoring devices (e.g., sensors) to determine whether or not the physiological event should be classified as an allergic reaction. In a specific embodiment, a determination of an allergic reaction triggers or causes the particle collection device to sample the ambient air in the user's local environment. In other words, the particle collection device is located locally in the user's local environment. For example, the particle collection device may be placed inside the user's house, outside the user's house (e.g., placed in user's backyard), office, or car. There can be multiple particle collection devices. The allergic reaction can then be associated with the particles collected at the time, date, and location of the allergic reaction to narrow and identify the types of particles that may contribute to the user's allergic reaction.

In particular, the analysis server, particle collection device, or both can identify airborne particles and, more specifically, allergenic particles such as pollen. The allergic reaction can then be correlated to one or more specific pollen types that have been collected and identified. This information can be used to help identify which allergens are responsible for a particular user's allergic reaction. In turn, a treatment plan can be developed to reduce or eliminate the occurrences of an allergic reaction.

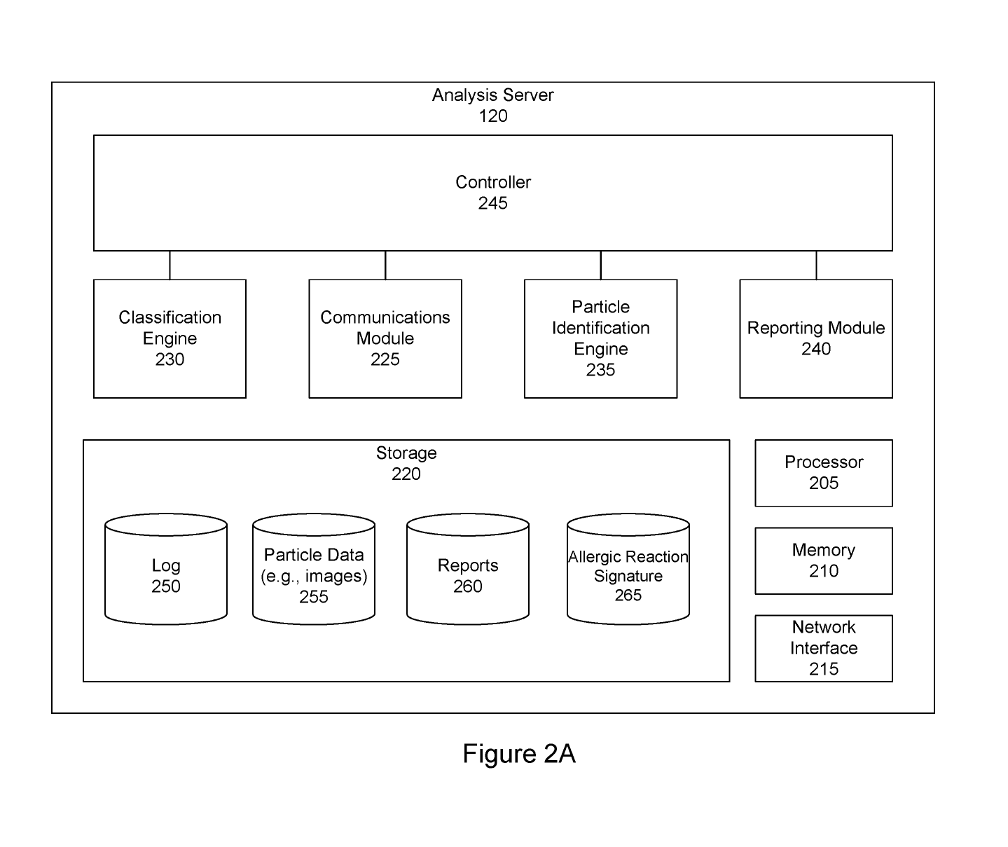

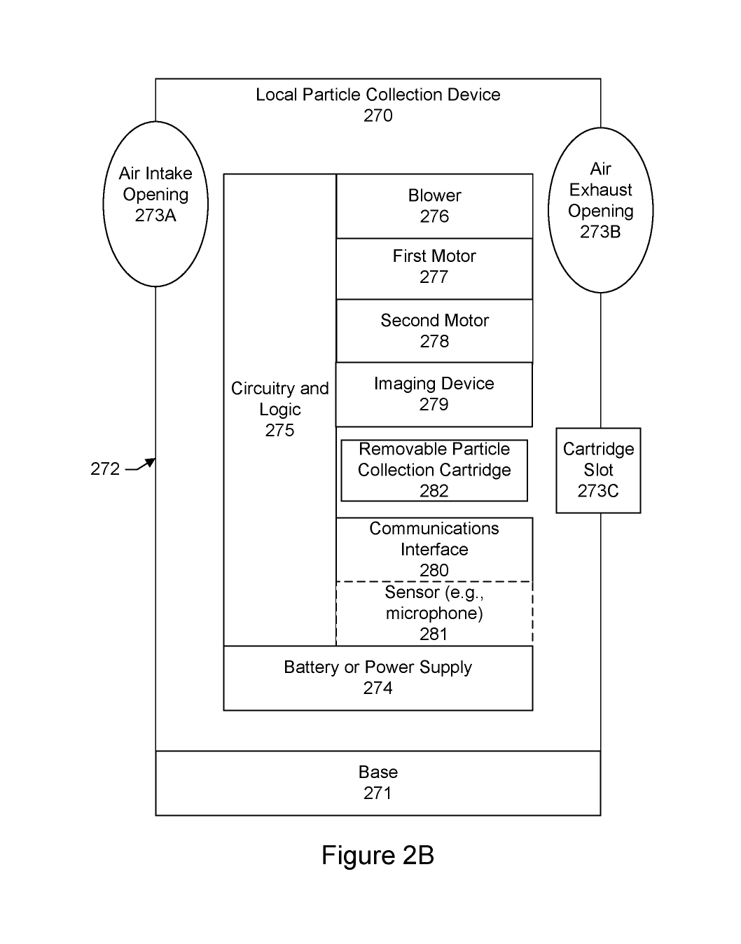

FIG. 2A shows a more detailed block diagram of the internal modules, components, or code components of the analysis server shown in FIG. 1 according to a specific embodiment. The analysis server may include a general purpose computer including hardware and software. For example, the analysis server may include a processor 205, memory 210, a network interface 215, and storage 220. The analysis server executes executable code (or computer-readable code) that embodies a technique or algorithm as described herein.

As shown in the example of FIG. 2A, the analysis server may include a communications module 225, a classification engine 230, a particle identification engine 235, a reporting module 240, and a controller 245. The storage may include a log 250, particle data repository 255, a reports database 260, and a database 265 storing an allergic reaction signature.

The controller is responsible for coordinating and orchestrating the activities of the various components and modules. The communications module is responsible for receiving physiological event data detected by the sensors, issuing instructions to the particle collection machine to collect particles, transmitting log and other data to the remote cloud server, and receiving updates or other communications from the remote cloud server.

The received physiological event data may include, for example, an audio or acoustic signal generated by a microphone when a user coughs or sneezes, activity data such as movement or motion data generated by an accelerometer attached to the user's wrist, strapped to the user's chest, or both. For example, motion data generated by the accelerometer may indicate that the user has raised their hand up to cover their mouth such as when coughing. Motion data may indicate a rapid series of chest movements such as when coughing. The event data may include video or images that may be captured by a camera and that may depict watery eyes, swollen eyes, or other indications of an allergic reaction.

The classification engine is responsible for analyzing the received physiological event data to determine whether the event should be classified as an allergic reaction. For example, the classification engine may compare characteristics of the audio signal against a known set of audio characteristics that are indicative of coughing, sneezing, or both. The characteristics of an audio signal that may be analyzed include oscillation period, amplitude, frequency, intensity, energy, uniform versus non-uniform energy distribution, pitch, duration, bandwidth, and the like, or combinations of these.

As another example, the classification engine may compare characteristics of the motion or movement data against a known set of movement characteristics that are indicative of coughing, sneezing, or both. Movement characteristics that may be indicative of coughing or sneezing include the user bringing their hand towards their face to cover their mouth during a cough or sneeze, the user's chest rapidly inhaling and exhaling, or both.

A known or predetermined set of characteristics may be referred to as a cough, sneeze, or allergic reaction signature. In a specific embodiment, determining whether or not a physiological event should be classified as an allergic reaction is based on evaluating two or more different types of data such as acoustic data and motion data. For example, acoustic data indicating a cough or sneeze that is accompanied by or contemporaneous with movement data indicating that the user raised their hand towards their mouth can strongly suggest that the event should be classified as an allergic reaction. Evaluating different types of data in combination can help to improve the accuracy of the classification.

In another specific embodiment, a single type of data is analyzed to make the determination of whether the physiological event should be classified as an allergic reaction. For example, an audio signal may be analyzed to determine whether the event should be classified as an allergic reaction and, in this specific embodiment, motion data may be excluded or may not be included in the analysis. In this example, microphones are used to detect the sounds and other sensors such as accelerometers, gyroscopes, and cameras may be omitted. Omitting or excluding other sensors helps to lower the cost of the system.

In a specific embodiment, upon the classification engine determining that the physiological event should be classified as an allergic reaction, the analysis server issues a request to the particle collection machine to collect particles that may be floating or present in the ambient air local to the user. In a specific embodiment, the particle collection machine remains dormant until it receives a request to sample the ambient air.

In another specific embodiment, the particle collection machine does not remain dormant and control samples are collected in the absence of an allergic reaction. In a specific embodiment, the control samples are collected periodically. For example, control samples may be collected every 5, 10, 15, 20, 25, 30, or 60 minutes, or at any other frequency as desired. Control samples may be collected continuously. Control samples may be collected during periodic intervals. For example, control samples may be continuously collected for a period of X minutes every Y minutes. As yet another example, sampling may be continuous with pollen containing air can be sampled a slow flow rate (e.g., in units of liters per minute) during control sample periods and at a faster flow rate when triggered. Sampling at a slow flow rate helps to conserve system resources. Sampling at a faster flow rate when an allergic reaction is detected helps to ensure that the airborne particle that might be responsible for the allergic reaction is collected.

The collection frequency or parameters are configurable such as by the user or administrator of the system. Frequent collections help to increase the amount of data available for analysis but can consume more resources as compared to less frequent collections. Configuring the collection frequency and collection parameters can be based on factors such as the location of the user, local weather or atmospheric conditions, time of year, time of day, and other factors. For example, if the user happens to be in a very dynamic, fluid, or changing environment such as outside in their backyard on a windy day, it may desirable to set a high collection frequency to ensure that any pollen that happens to be carried into the user's backyard is captured.

The collection of control samples helps to address an effect that this patent application refers to as the “priming effect.” In this application, “prime” refers to any immune system mechanism by which past allergen exposure sensitizes or “primes” the immune system to react or react more strongly to a present exposure to allergens. This includes mechanisms in which IgE antibodies are produced and arm mast cells. This also includes mechanisms involving migration of immune system cells to the exposed tissue. In some cases, the IgE mechanisms may be referred to as “induction of sensitivity” while the term “priming” may be reserved for mechanisms that also involve migration of immune cells. This patent application uses the terms “prime” and “sensitize” as synonyms implying a relationship between past exposure and reactions to present exposure without regard to the details of immune system mechanisms for purposes of clarity in illustrating the principles and aspects of the system.

The “priming effect” is a key aspect of the users' response to allergens. Even when a user's immune system has a long-term memory that it is allergic to a particular allergen, the user does not necessarily immediately react with observable symptoms when exposed to the allergen. First the immune system needs to be “primed” or sensitized to the allergen. Modern scientists have uncovered a number of physiological mechanisms that explain the priming effect.

For example, when memory immune cells recognize an allergen, they may trigger immune system production of “IgE” immunoglobulin anti-bodies that in turn may be absorbed by the cellular membranes of immune mast cells in the nose. This may enable these immune cells to recognize the particular allergen, and hence set the stage for an allergic reaction. In other words, a user's allergic reaction is not only in response to an immediate triggering exposure to an allergen, but also in response to earlier sensitizing or priming exposure to either the same or a related allergen. The system can help to identify both triggering allergens and priming allergens. Further discussion is provided below.

The particle collection machine samples ambient air as a means to estimate user exposure to airborne allergens such as pollen as a function of time. Timing information, and its relationship to the physiology of allergic reactions, plays a key role in the use of particle collection machine data. An understanding of the timing characteristics of the immune system's reaction to airborne allergens is needed to appreciate the further embodiments described below.

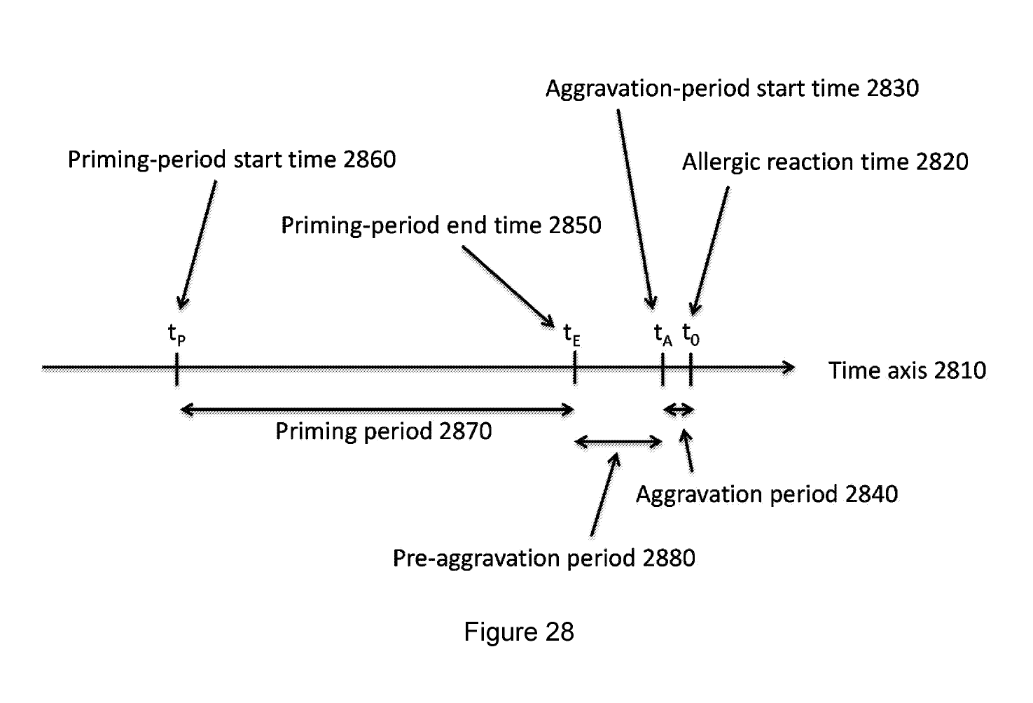

Referring to FIG. 28, an allergic reaction timing diagram is shown. Let to represent an allergic reaction time 2820 at which a user starts suffering from an allergic reaction. The start of user suffering from allergic reaction is likely to be due to what in medical science is known as “early phase” symptoms rather than “late phase” symptoms. This suggests considering IgE mediated immune system mechanisms as a specific example. The immediate cause of the allergic reaction was the inhalation of an allergenic pollen or other airborne allergen that was recognized by immune cells armed with IgE anti-bodies specific to the inhaled allergen. These IgE-armed immune cells initiated a chain of physiological reactions resulting in symptoms experienced by the user. Allergens recognized by such IgE-armed immune cells may be referred to as “aggravating allergens,” such as an “aggravating pollen.” More generally, aggravating allergens are allergens that are the immediate cause of an allergic reaction.

Some time passes between the inhalation of the aggravating allergen and the resulting symptoms. Hence the allergic reaction starting at time t₀ must be due to exposure to the aggravating allergen sometime before time t₀. However, there is a limit to how long the onset of symptoms can be delayed with respect to the exposure. Let t_{A} be the earliest time for which exposure to the aggravating allergen could lead to symptoms that do not occur until time t₀. The period of time between time t_{A} and time t₀ may be referred to as the aggravation period 2840. The aggravation period 2840 is bounded by the aggravation-period start time 2830 (t_{A}) and the allergic reaction time 2820 (t₀).

In some cases, the duration of the aggravation period 2840 is generally not precisely known and might vary with the type of allergen or vary from user to user. Nevertheless, it is clearly a time period very short compared to one day and long compared to a second. Medical science research has established reasonable estimates of the duration of aggravation period 2840 to include the range from a few minutes to a half hour. For example, the aggravation period may range from about 2 minutes to about thirty minutes. This includes, for example, 5, 10, 15, 20, 25, 29, or more than 29 minutes. The aggravation period may be less than 2 minutes.

The allergic reaction described in the previous paragraph is most likely to occur if the user's immune system has been primed, that is, only if the user's body contains immune cells armed with IgE anti-bodies specific to the aggravating allergen. Such priming is the result of an earlier exposure to an allergen that is either the same as the aggravating allergen or is another allergen that cross-reacts with the aggravating allergen. The allergen that primes the immune system may be referred to as the “priming allergen.” The priming allergen may or may not be the same as the aggravating allergen.

When a priming allergen is recognized by an immune system memory cells, the memory cells cause the user's immune system to start manufacturing IgE anti-bodies specific to the priming allergen. Once manufactured, these IgE anti-bodies make their way to the immune cells and play a key role in the aggravation period 2840. There is a time delay between user exposure to a priming allergen and the arming of immune cells with corresponding IgE anti-bodies.

As a result, there is a time after which it is too late for a priming allergen to have contributed to the chain of events leading to an allergic reaction at time t₀. Let t_{E} represent this priming-period end 2850. In some cases, it is not precisely known how far in the past the priming-period end 2850 occurs, that is, the quantitative value of the time difference (t₀−t_{E}) is not precisely known. The value may depend on both the allergen and the user. Nevertheless, it is clearly a time period very short compared to a week and long compared to an hour. Medical science research has established reasonable estimates for the time difference (t₀−t_{E}) to include the range from 12 hours to 2 days. For example, the priming-period end time 2850 may range from about 12 hours to about 2 days. This includes, for example, 15, 20, 25, 30, 35, 40, 45, or more than 45 hours. The pre-aggravation period may be less than 15 hours.

The priming effect is temporary. After exposure to priming allergens end, the corresponding priming effect fades with time. In biochemical terms, after exposure to priming allergens end, manufacture of priming-allergen specific IgE anti-bodies cease and eventually previously manufactured IgE anti-bodies degrade and disappear. As a result, there is a time before exposure to priming allergens that is too early to explain the primed state of the immune system at the allergic reaction time t₀. Let t_{P} represent this time that may be referred to as the priming-period start time 2860.

Exposure to priming allergens during the priming period 2870, that starts with the priming-period start time 2860 and ends with the priming-period end time 2850, can explain the primed state of the user's immune system at allergic reaction time t₀ (or more precisely can explain the primed state of the user's immune system during the aggravation period 2840). The duration of the priming period 2870 is equal to (t_{E}−t_{P}). In some cases, it is not precisely known how long this priming period 2870 is. The value may depend on both the allergen and the user. Nevertheless, it is clearly a time period short compared to a month and very long compared to a day. Medical science research has established reasonable estimates for the time difference (t_{E}−t_{P}) to include the range from one day to one month. For example, the priming period may range from about 3 hours to about 1 month. This includes, for example, 1, 2, 4, 6, 15, 20, or 30 days. The priming period may be less than 1 day.

The time period between the priming-period end time 2850 and the aggravation-period start time t_{A} may be referred to as the pre-aggravation period 2880. Allergens inhaled by the user in this pre-aggravation period 2880 are too late to be the priming allergen contributing an allergic reaction at time t₀ and too early to be the priming allergen contributing to an allergic reaction at time t₀. Lack of allergic reaction due the pre-aggravation period 2280 provides evidence that any allergens inhaled during the pre-aggravation period 2280, even if also present in the aggravation period 2240, are not the guilty aggravating allergen. Allergens present in the aggravating period 2240 but not present in the pre-aggravating period 2880 are the prime suspects for aggravating allergens.

In some embodiments, the conclusion of the previous paragraph is tentative or preliminary and the user is prompted by the system to answer questions about medication. It is possible that the user had been taking medication that suppressed allergic reaction symptoms in the pre-aggravation period 2880 and then the medication wore off during the aggravation period 2840. In this scenario, allergens detected in the pre-aggravation period 2880 remain candidates for being aggravating allergens.

FIG. 28 and the associated discussion above is idealized in the sense that transitions at priming-period start t_{P}, priming-period end t_{E} and aggravation-period start t_{A} are naïvely presented as sharp boundaries. This idealization is presented for purposes of clarity. In reality, the boundaries are fuzzy and the transitions are more gradual. For example, assuming a value the priming-period t_{P} of two weeks, common sense tells us not to expect that an exposure to a priming allergen two weeks before allergic reaction time t₀ to be 100 percent effective while an exposure to a priming allergen two weeks and one minute before allergic reaction time t₀ to be totally ineffective.

In a specific embodiment, a sophisticated mathematical model is provided where the fading of the strength of the priming effect with increasing times into the past can be represented by a factor of exp{−(t₀−t)/τ} where t is the time of priming allergen exposure and τ is an exponential time constant. However, for an understanding of the basic principles it is not necessary to delve into such mathematical details. Thus, it should be appreciated that FIG. 28 and associated discussion has been simplified for clarity of presentation and further mathematical refinement can be made without departing from the scope of the present disclosure.

The above discussion of FIG. 28 concerns the human immune system and considered inhaled allergens. When applying this science to particle collection machines, it should be kept in mind that allergen exposures that are measured by particle collection machines may provide imperfect estimates of exposures of the user to inhaled allergens.

To give a clear example where it is important to distinguish between measured allergen exposure and true allergen exposure, consider a user that places a pollen collection machine in a combined kitchen-living room, but sleeps in a well-separated bedroom. Furthermore, let us assume allergenic pollen from outdoors makes its way into the kitchen-living room but not the bedroom. After waking up from a good nights sleep, the user leaves the bedroom and enters the kitchen-living room, and then shortly thereafter has an allergic reaction at allergic reaction time 2820.

In this case the user did not inhale the aggravating allergen until entering the kitchen-living room. The user was not exposed to the aggravating allergen in the pre-aggravation period 2880. Nevertheless the pollen collection machine did measure the presence of the aggravating pollen in the pre-aggravation period 2880. To correctly identify the aggravating allergen, one must allow the possibility that the aggravating allergen is detected during the pre-aggravation period 2880 even if true exposure to an allergen in the pre-aggravation period is reason to eliminate the allergen as the aggravating allergen.

Referring back now to FIGS. 1 and 2A, in a specific embodiment, the particle collection machine captures color images of the collected particles. In a specific embodiment, the color images can then be provided or transmitted across the network to the analysis server. The particle identification engine is responsible for reviewing the particle data received from the particle collection machine and identifying the particles. The identification engine may identify particles such as pollen based on color, shape, size, structural features, time of year, weather, geographical location, other factors, or combinations of these.

In another specific embodiment, the analysis functions are performed at or by the local particle collection device. Any competent system or technique may be used to identify or discriminate the collected particles. Some of these systems and techniques are discussed in U.S. provisional patent applications 62/173,280, filed Jun. 9, 2015 and 62/210,253, filed Aug. 26, 2015. These patent applications are assigned to the same assignee as this patent application and are incorporated by reference. Any of the systems and techniques discussed in these patent applications for imaging and identifying particles such as pollen are applicable to the systems and techniques including the particle collection device in this application.

The reporting module acts as a user interface for displaying reports and results from the analysis. There can be an electronic screen coupled to the analysis server to display information such as a time, date, and location of a user's allergic reaction and identifications of particles that were collected at the time, date, and location of the user's allergic reaction, identification of the aggravating allergen, identification of the priming allergen, and so forth. The reporting module may generate emails including the results, display the results in a news feed, include the results in a text message, or combinations of these.

The log stores records of detected allergic reactions and particle collection activity. Entries in the log may record, for example, parameters or metadata associated with an allergic reaction and a particle collection including the collection of control samples. The parameters may include a recording of time, date, location, atmospheric conditions (e.g., temperature or humidity levels), or combinations of these.

The data logs generated by the system may include a timestamp of the allergic reaction (e.g., time and date of allergic reaction), a recording of the physiological event associated with the allergic reaction (e.g., an audio recording of a cough or sneeze), a geographical location of the allergic reaction (e.g., global positioning or GPS coordinates such as latitude and longitude), an indication of whether the allergic reaction occurred while the user was in an outside or inside environment (e.g., outdoors or indoors), weather conditions associated with the allergic reaction (e.g., temperature, humidity, wind speed, or wind direction), particle analysis data, or combinations of these.

The particle data repository includes images, pictures, or photographs of particles collected by the particle collection device. In a specific embodiment, the images are color images. The particle analysis data may include data derived from analyzing the locally collected particles associated with the allergic reaction and data derived from analyzing locally collected control samples of particles that may have been collected before or prior to the allergic reaction.

The particle analysis data may include pictures or images of collected pollen including color pictures, particle size, particle characteristics and features, particle count (e.g., a number of grains of pollen in a cubic meter of air), or combinations of these.

The reports database stores reports generated by the reporting module. A report may include, for example, the time and date of an allergic reaction, a cross-reference to an identification of particle (e.g., pollen) types collected at the time and date of the allergic reaction, an identification of particle types collected during a rolling time period prior to the allergic reaction, or combinations of these. A particle collected at or near the time and date of the allergic reaction may be referred to as an aggravating allergen. A particle collected during the rolling time period prior to the allergic reaction is a candidate for what may be referred to as a priming allergen. It should be appreciated that not all types of pollen observed in the priming period contribute to an allergic reaction.

The allergic reaction signature database stores characteristics of an allergic reaction. For example, an acoustic signature may be stored where the data includes sounds or acoustic parameters and corresponding values associated with coughing, sneezing, or both. A motion signature may be stored where the data includes motion data associated with coughing, sneezing, or both. Images may be stored where the image data includes facial images depicting symptoms of an allergic reaction (e.g., watery and irritated eyes).

In a specific embodiment, the allergic reaction signature is derived from data supplied by the user. For example, in a specific embodiment, a setup and configuration process includes prompting the user to simulate an allergic reaction. The system receives and records the simulated allergic reaction which may include recording audio data, motion data, facial images, or combinations of these as the user simulates the allergic reaction. The system may derive or generate an allergic reaction audio signature based on the audio data, an allergic reaction motion signature based on the motion data, an allergic reaction image signature based on the image data, or combinations of these.

Referring back now to FIG. 1, the data received, generated, or stored by the analysis server may be transmitted to the remote cloud server. For example, the logs, particle data, reports, allergic reaction signatures, or combinations of these may be transmitted from the analysis server to the remote cloud server. The remote cloud server provides a central location for storing data associated with the various users of the system. The data stored at the remote cloud server may include user information (e.g., name, address, or age), medical information or records (e.g., known allergies, current medication, or past medication), local environment information (e.g., the type of vegetation or plants, trees, shrubs, or flowers present in or near the user's house, yard, or office), satellite or street imagery of the user's house or office that may be used to help identify pollen sources, data logs generated by the system, or combinations of these.

The system can review, analyze, and aggregate the data to provide detailed allergy forecasts, generate customized pollen calendars, and so forth. These reports may be provided through a subscription-based data service. Subscribers may receive customized alerts or allergy forecasts so that they can plan accordingly (e.g., avoid outdoor activities or wear a dust mask).

In a specific embodiment, the remote cloud server includes a central management server. The central management server provides a central management of all the components in the system. For example, the central management server provides a single location to view system status such as the status of the various particle collection devices that may be deployed in various user environments. Firmware updates may be distributed from the central management server to the particle collection devices, analysis servers, or both. For example, updates or patches to the particle or pollen identification and analysis algorithms may be distributed from the remote cloud server.

As discussed above, the particle collection device is a mechanical device that collects physical airborne particles that may be floating in the user's ambient or local environment. The collection device may perform an analysis or initial analysis or identification of the collected particles. The particle collection device is a network-enabled device and can receive requests, commands, and instructions over the network such as from the analysis server, and transmit responses and other data across the network such as to the remote cloud storage server. Further discussion is provided below.

It should be appreciated the system shown in FIGS. 1 and 2A are merely illustrative. The blocks shown in FIGS. 1, 2A, and 2B (discussed below) can be functional entities, rather than structural, and there can be many different hardware configurations that can perform the functions shown and described. For example, the functions of the analysis server including the analysis logic and hardware components such as the processor, storage, memory, and so forth may be built into or integrated with the local particle collection machine. The functions of the analysis server may be built into an allergic reaction monitoring device with sensor. The local particle collection machine itself may include sensors such as a microphone to detect coughs, sneezes, and so forth.

In another specific embodiment, the analysis server is implemented as a hardware device that is placed in the user's local environment and that is separate from the local particle collection machine. For example, the analysis server and the particle collection machine may both be located in the user's house or office. The system may include first and second cabinets. The first cabinet houses internal components of the analysis server and may be referred to as a base station or local base station. The second cabinet, different from the first cabinet, houses internal components of the particle collection machine. The analysis server and particle collection machine may be connected to a local area network for communication with each other.

In another specific embodiment the analysis server is remote from the local particle collection machine. For example, the analysis server and remote cloud server may be located in a data center. In this specific embodiment, the local particle collection machine may include hardware and software that allows for communications with the analysis server, remote cloud server, or both over a wide area network (e.g., the Internet). The remote server may participate in, for example, determining whether the user suffered an allergic reaction, identifying particles, or both. The remote server may have access to resources such as data logs, compute resources, and so forth that may not be available in the system components local to the user. Thus, for example, pollen images may be transmitted to the remote server for pollen identification and analysis. Physiological event data may be transmitted to the remote server to determine whether the user suffered an allergic reaction.

FIG. 2B shows a block diagram of a local particle collection device 270 according to a specific embodiment. The particle collection device includes a housing 272 mounted to a base 271. The housing includes an air intake opening 273A, an air exhaust opening 273B, and a cartridge slot opening 273C. There can be a door connected to the cartridge slot opening via a hinge. The door can open into the cartridge slot. Shown inside the housing are a battery or power supply 274 which is connected to circuitry and logic 275 which in turn is connected to a blower 276, first motor 277, second motor 278, imaging device 279, communications interface 280, and sensor 281 (e.g., microphone). The sensor is shown in broken lines to indicate that it is not included in some embodiments.

Further shown in FIG. 2B is a particle collection cartridge 282. The particle collection cartridge includes a reel of tape media. The tape media is wound about the reel and includes an adhesive to collect airborne particles (e.g., pollen). The collection cartridge is removable from the collection device. That is, a user can remove the cartridge from the collection device without breaking or destroying the device. There can be an eject button that the user can press to eject the cartridge from the particle collection device. For example, when the collection cartridge is full (or as desired), the user can remove the collection cartridge from the collection device through the cartridge slot opening. The user can then install a new collection cartridge by inserting the new collection cartridge into the collection device through the cartridge slot opening. The user can then mail the removed collection cartridge—which contains the collected airborne particles—to a laboratory for a further in-depth analysis.

The particle collection device may include an electronic screen to display a status associated with operations of the particle collection device (e.g., “collection cartridge tape 80 percent full,” “analyzing particles,” “device error,” “transmitting data to remote cloud server,” “firmware update in progress, please wait,” and so forth). There can be status lights such as LED status indicators. The particle collection device may include an input device such as a keypad through which the user can power the device on or off, configure various settings and parameters such as collection frequency, other settings, and so forth. Instead or additionally, at least some settings may be configured remotely.

The blower may include a fan and is responsible for creating a vacuum in which air is sucked into the collection device thorough the air intake opening. A flow path of air is directed to the particle collection cartridge. Particles that may be floating or suspended in the air are trapped by the adhesive tape of the particle collection cartridge. The air then exits the collection device through the air exhaust opening.

The first motor operates to rotate the housing of the collection device about the base. The collection device may include an airflow sensor or airflow direction sensing unit that detects a direction of the flow of the ambient air. Based on the direction of the airflow, the first motor can rotate the collection device to orient or align the air intake opening with a direction of the flow of the ambient air. Instead or additionally, the first motor may be configured to continuously or periodically rotate to obtain good representative samples of the ambient air.

The second motor engages the reel of the tape media to unwind the adhesive coated tape media. For example, as airborne particles such as pollen become trapped in a portion of the adhesive coated tape, the second motor can unwind the reel to expose a new portion of the adhesive coated tape upon which new airborne particles can be collected.

The second motor is further responsible for advancing the tape containing the trapped particles to the imaging device. The imaging device captures images (e.g., pictures) of the trapped particles for analysis and identification.

The communications interface is responsible for communications with, for example, the allergic reaction monitoring device, analysis server, remote cloud server, or combinations of these. The communications interface may include an antenna for wireless communication. The sensor (e.g., microphone) may be as described above.

As discussed, in a specific embodiment, the functions and capabilities of the analysis server may be integrated into a local particle collection device. For example, the logic of the particle collection device may include logic to classify a physiological event, detect an allergic reaction, identify captured particles, store particle logs and other data, generate reports and notifications, and so forth. Integrating the capabilities of the analysis server into the local particle collection device helps to reduce the number of physical components that are deployed in the user's local environment.

In another specific embodiment, the analysis server and local particle collection device are separate from each other. Having the analysis server and local particle collection device separate from each other can lower the overall cost of the system in cases where, for example, multiple particle collection devices are deployed. In this specific embodiment, each locally deployed collection device can rely the same analysis server for particle identification and analysis.

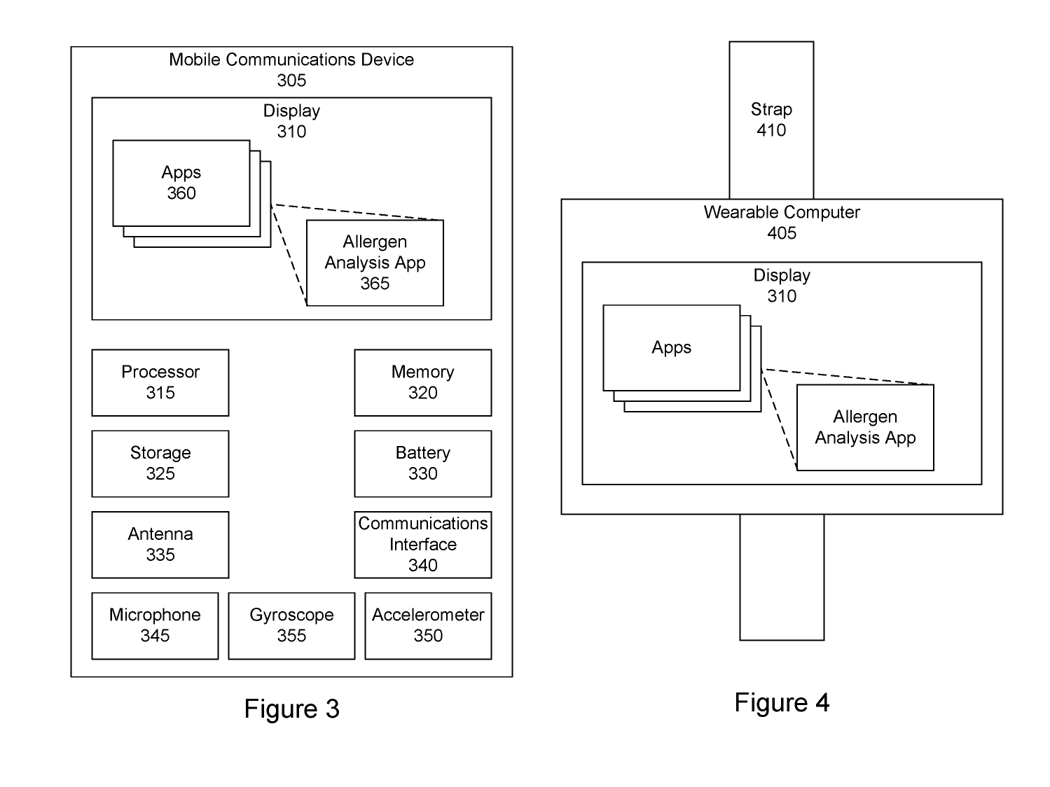

FIG. 3 shows a block diagram of another specific embodiment of the system. In the example shown in FIG. 3, there is a mobile communications device 305 such as a smartphone. The mobile communications device is designed to be portable and may be carried by a user such as in the user's pocket or purse.

The mobile communications device may include a display 310, processor 315, memory 320, storage 325, battery 330, antenna 335, communications interface 340, and one or more sensors such as a microphone 345, accelerometer 350, and gyroscope 355.

The mobile communications device can execute mobile application programs 360 or “apps.” Such apps may be available for download on application marketplaces or other websites such as the Apple App Store, Google Apps Marketplace, Amazon Appstore, and others. In a specific embodiment, functions of the analysis server are implemented in an allergen analysis app 365. Users can download and install the app onto their mobile devices. The allergen analysis app includes logic, code modules, or algorithms to classify physiological events detected by the sensors of the mobile device, communicate with the local particle collection device, receive particle data from the collection device, identify the particles, and communicate with the remote cloud server.

The allergen analysis app can be paired with a sensor (e.g., microphone) that is external to the smartphone and that may be clipped to the user's shirt or ear. The communication link between the sensor and the allergen analysis app can be a wired or wireless communication link (e.g., Bluetooth or other wired or wireless communication standard). This allows for detecting events such as coughs and sneezes even though the mobile communications device may be in the user's pocket or purse.

Audio detection of allergic reactions and an associated app or code component may be integrated into voice recognition systems that also serve other purposes. For example, voice recognition or interface services such as Apple's Siri, Amazon's Echo, Microsoft Window's Cortana, and so forth may be upgraded to include audio detection of allergic reactions as described herein.

In a specific embodiment, the app allows the user to issue an on-demand or manual request to the particle collection device to conduct a sampling. The on-demand sampling request can be used in cases where the symptoms of an allergic reaction are not detected by the sensors. For example, in some cases, an allergic reaction may be limited to an itchy nose or throat. Depending upon the type of sensor being used, these symptoms may not be detected.

FIG. 4 shows a block diagram of another specific embodiment of the system. In this specific embodiment, there is a wearable computer 405. In the example shown in FIG. 4, the wearable computer includes a strap 410 having a fastening mechanism such as a buckle or Velcro. The wearable computer can be strapped to the user's wrist or chest. The wearable computer can include hardware and software similar to that shown in FIG. 3 and described in the discussion accompanying FIG. 3. For example, the wearable computer may include a display, apps including an allergen analysis app, processor, memory, and one or more sensors (e.g., microphone, accelerometer, or gyroscope). The wearable computer may be implemented as a smartwatch. The wearable computer may be implemented as a wearable tracking or monitoring device that may or may not include a display. One of ordinary skill in the art would recognize other variations, modifications, and alternatives.

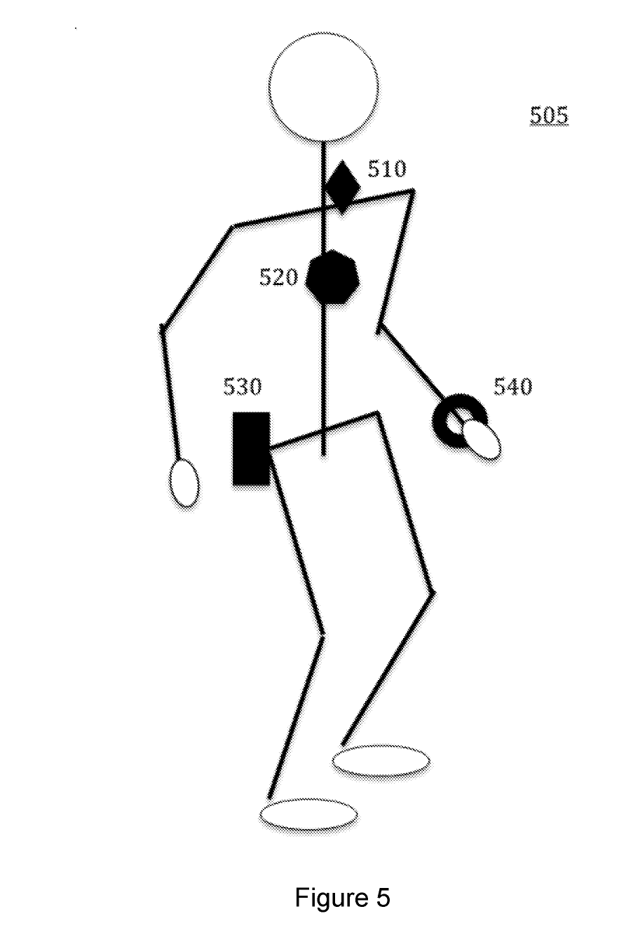

FIG. 5 shows a sketch 505 that illustrates several options for wearable allergic reaction monitors. One option includes a clip-on device 510 like microphones often worn by public speakers at talks. Another option includes a pendant device 520 suspended from a string or lanyard around the user's neck. Yet another option includes a device 530, such as a smartphone with an appropriate app (e.g., allergen analysis app as described above), that is held snug against the user's body in a pants pocket. The last option illustrated in the figure, includes a device 540 worn around the user's wrist much like a wristwatch. Sketch 505 illustrates that there are many options for wearable allergic reaction monitors, but, although not shown in the figure, there can be other options. There is presently a great deal of entrepreneurial and technology innovation in the field of wearable electronics, particularly for health monitoring purposes; as a result the future may well see increasingly low-cost and convenient means to monitor the allergic reactions of users. Such monitors of user physiology might even include implantable sensors that have more direct access to biochemical markers of user immune reactions.

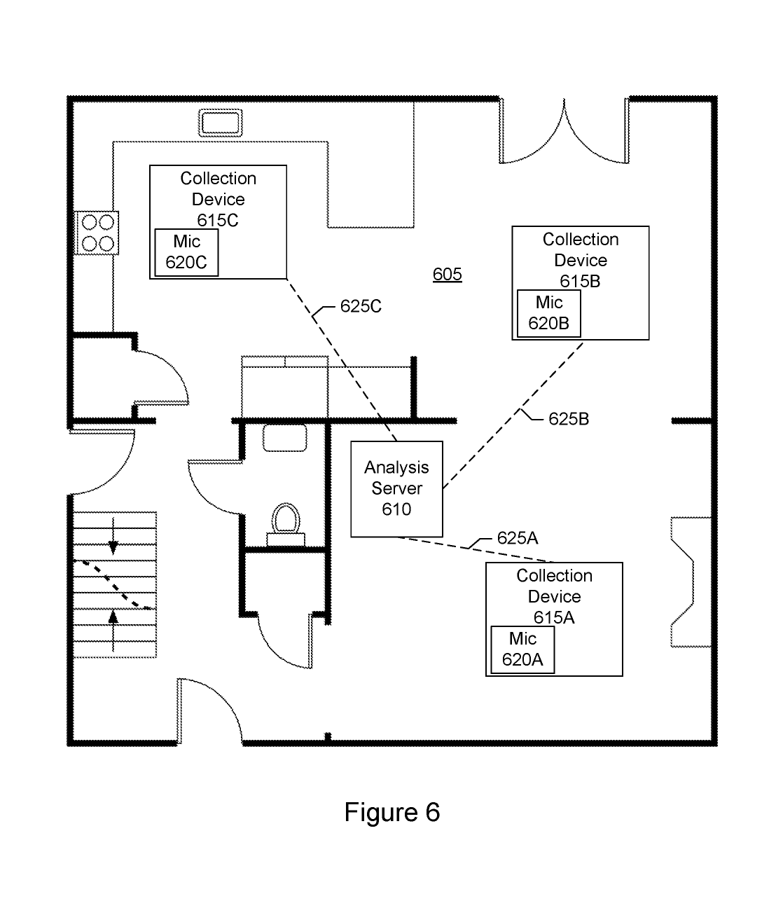

FIG. 6 shows a floor plan 605 of a house illustrating another option for the deployment of the system according to a specific embodiment. In this specific embodiment, there is an analysis server 610 located in the living room of the house and first, second, and third particle collection devices 615A, B, and C located in the living room, dining room, and kitchen, respectively. Particle collection devices 615A-C include microphones 620A, B, and C, respectively. The particle collection devices and analysis server are connected via communication links 625A, B, and C. The communication links can be wired or wireless communication links.

In this specific embodiment, a sensor (e.g., microphone) of a collection device detects sound. The collection device sends an audio signal of the sound to the analysis server for analysis. The analysis server analyzes the sound to determine whether the sound should be classified as an allergic reaction. If the sound should be classified as an allergic reaction, the analysis server sends an instruction to the respective collection device to sample the ambient air.

In cases where multiple sensors, (e.g., microphones) and collection devices have been deployed, the collection device nearest or in closest proximity to the user can be instructed to collect the sample. The system can determine which collection device is nearest the user based on the intensity of sound (e.g., coughing or sneezing sounds) detected at the microphones.

For example, a first subset of microphones may be paired to a first collection device located in a first room (e.g., dining room) of the user's house. A second subset of microphones may be paired to a second collection device located in a second room (e.g., living room) of the user's house. When the user coughs or sneezes, the sound may be detected by both the first and second subset of microphones. Based on the intensity or level of sound detected at each of the first and second subset of microphones, one of the first or second collection devices performs a sampling. If the level of sound detected at the first subset of microphones is greater than the level of sound detected at the second subset of microphones, the first collection device performs the sampling. Alternatively, if the level of sound detected at the second subset of microphones is greater than the level of sound detected at the first subset of microphones, the second collection device performs the sample.

In another specific embodiment, a sensor may be embedded in a household appliance such as a refrigerator, microwave, oven, television, radio, lighting fixture, alarm clock, or any other type of physical object or appliance. The appliance including the sensor includes electronics and network connectivity which enables the appliance to communicate with other devices such as the particle collection device.

In another embodiment, the particle or pollen sampler is integrated into the personal automobile of the user. In some respects, automotive applications have particular advantages for the system. The automobile's electrical system can be used to power the pollen sampler, the allergic reaction monitor, or both. When an allergic reaction is detected, or if a type of pollen previously recognized as allergenic for the user is detected, the automobile's air circulation system may choose to recirculate cabin air rather than draw in more pollen laden external air, or may take stronger measures to filter the air. Furthermore, within a moving automobile, the system disclosed herein may be able to sample a larger quantity and diversity of air samples than a system confined to a user's home.

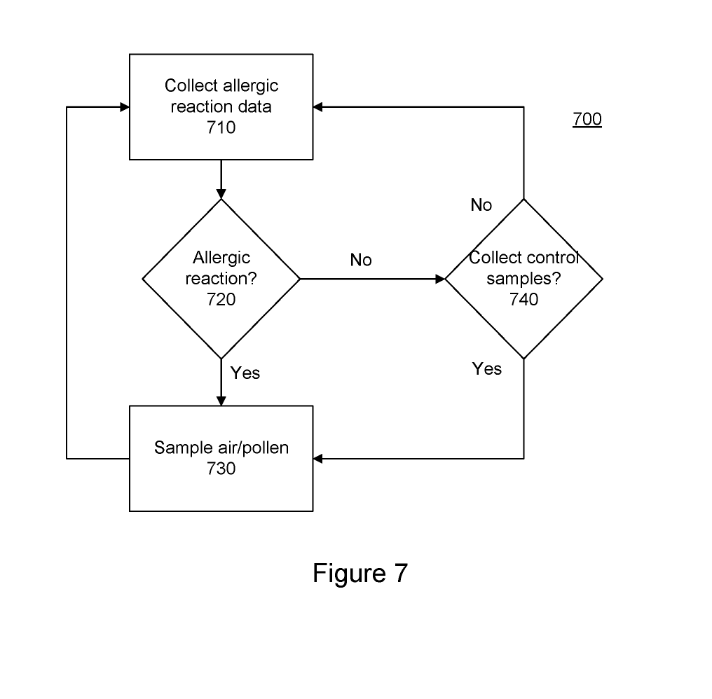

FIG. 7 shows a flow 700 of a process of the system according to a specific embodiment. Some specific flows are presented in this application, but it should be understood that the process is not limited to the specific flows and steps presented. For example, a flow may have additional steps (not necessarily described in this application), different steps which replace some of the steps presented, fewer steps or a subset of the steps presented, or steps in a different order than presented, or any combination of these. Further, the steps in other embodiments may not be exactly the same as the steps presented and may be modified or altered as appropriate for a particular process, application or based on the data.

This flow illustrates the top-level conceptual building blocks of the system. In a step 710, an allergic reaction monitor associated with a user collects data relevant for monitoring possible allergic reactions of the user. In a step 720, a decision is made whether or not the user is suffering from an allergic reaction (such as sneezing, coughing, or both). If the decision is “yes,” then air is sampled for pollen by a pollen sampler (step 730). If at step 720 the decision is “no,” then the process may transition back to step 710 for further collection of allergic reaction data.

Alternatively, as is illustrated in the figure, a “no” decision at step 720 may lead to a transition to a step 740 where a decision is made whether or not to collect control samples of pollen in the air when the user is not suffering an allergic reaction. For example, collect allergic reaction data 710 might correspond to capturing 5 seconds of audio recorded with a microphone every 5 seconds, and if no allergic reactions are being detected during the 5-second recordings then a “no” decisions are step 720 leading to a step 740 decision every 5 seconds. If it is determined that sampling once every 5 minutes is sufficient for control samples, then the decision at step 740 may be “Yes” once out of every 60 decisions followed by 59 “No” decisions. Of particular diagnostic interest are pollen types that are collected by the pollen sampler during allergic reactions, but not collected in control samples when the user has no allergic reaction. In a specific embodiment, a decision at box 740 to collect control samples refers to whether or not a current time is between control sample collection periods.

Step 720 is accomplished with the aid of an allergic reaction monitor that is worn by the user, is located in proximity to the user, or both. The allergic reaction monitor may use any type or combination of types of sensors to detect any user symptom or combination of symptoms of allergic reactions on the part of the user.

Consider, as an example, the following scenarios.

While many physiological parameters, such as body temperature, heart rate, blood oxygen levels, ECG signals, etc. may have some correlation with allergic reactions, audio sounds and body movements associated with coughing, sneezing, or both are of particular interest. This is both because they are common symptoms of allergic reactions and because of the potential for cost-effective and non-invasive sensing options. Common low-cost microphones on the user or in proximity to the user can detect audio sounds of coughing, sneezing, or both. Accelerometers such as commonly included in smartphones and wearable computers, when placed on the user may detect user motion.

In one specific embodiment, the allergic reaction monitor is comprised of an accelerometer and gyroscope for capturing movement (when the wearer is mobile/moving about, or standing still), an audio-recording microelectronic device (microphone) such as a voice coil or piezo-inductive device, memory for storing data, microcontroller, battery for powering the device, and a component such as a Bluetooth or WiFi for transmitting the trigger signal and data outside the device to an allergen detector such as a pollen counter or detector machine.

In a specific embodiment, the allergic reaction monitor device system may use one or a combination of techniques to identify the individual and when he or she is coughing or sneezing against any potential false positives before a trigger signal is sent. The first technique includes capturing the user's individual cough and sneeze signature. Similar to a fingerprint or eye retina, each person produces unique coughing sounds and patterns. This may be accomplished by first having the user cough and sneeze at various levels of loudness to which they are familiar. The device and accompanying software resolves and stores the unique sound frequencies associated with said user along with typical amplitudes, etc. and stores this in the device itself or a computing device with this software.

The second technique involves proximity where the accelerometer and gyroscopes are used to determine if the person wearing the device is moving or running, or standing still and when worn on a wrist it can trace an arm moving continuously towards the user's head and nose and the microphone will hear an amplification in cough/sneeze sound by being closer. When worn as a pendant close to the chest, the accelerometer and gyroscope can detect signals that when processed by the system indicate a unique fast and short shaking along with a muffled noise detected by the microphone.

The third technique includes adaptive algorithms whereby the more a person uses it the more it learns over time and continues to adjust and fine-tune the unique coughing and sneezing signatures. For example, as a person begins to develop a cold the device can see how their signature begins to slowly drift over a couple of days to a slightly lower frequency and pitch characteristic of congested lungs and respiratory airwaves. This would prevent the device from sending false triggers or completely missing some over the time a user had congested airwaves.

The allergic reaction monitor device could be either worn by user (custom device or smartphone with app), installed in the users environment (e.g., room of house or passenger compartment of an automobile), or both. In situations where the device is embedded in the environment, the gyroscopes and accelerometer are disabled from use, or simply not included in the device design, and detection may rely exclusively in detecting the unique cough and sneeze signatures of the particular user in that environment.

In a specific embodiment, when the devices or system matches a series of coughs, sneezes, or both from a user it sends a trigger signal to the collection device (e.g., pollen sampler). The pollen sampler is a device that samples ambient air and collects pollen that the air may contain. There may be one or more than one pollen sampler. The pollen sampler or samplers in proximity to the user are triggered to collect samples such as by the allergic reaction monitor device or analysis server.

Optionally the pollen sampler not only collects pollen samples, but also communicates with other devices, detects and analyzes the types of pollen samples, or combinations of these. For example, the nearest pollen sampler may also comprise a pollen detection/allergenic detection device to collect an air sample, provide feedback to the user about the allergens currently in the air, or both.

These could be:

1) A pollen/allergenic capturing device equipped with a camera based system for pollen detection on a tape substrate or glass or plastic slide with or without a sticky adhesive surface. In a specific embodiment, a cartridge having a tape substrate for pollen capture is provided. Optionally the camera may determine three-dimensional shapes of pollen via capture of images at various focal plane depths.

2) A pollen/allergenic capturing device that scans particles real-time in the air as it passes through various optical sensors that collect allergenic particles' unique spectral signatures. Optionally, the spectral signatures include fluorescence spectral signatures.

3) A pollen/allergenic capturing device that traps particles from the air for further bio-assay analysis and classification.

4) “Virtual pollen sampling” via processing of information on the users' location and information on the internet or “cloud” that may be predictive of users' pollen exposure. Such internet/cloud information may include local weather data, local geography, information about blooming seasons of various sources of pollen, and so forth. Furthermore, virtual pollen sampling may also be provided by a pollen-monitoring service company operating a fleet of drones carrying pollen monitors. Such drones may be, for example, unmanned aerial vehicles or self-driving automobiles. In some embodiments, such drones may respond to a user's allergic reaction by altering their paths to move into closer proximity to the user.

Each of these devices may provide the results that may be stored and may be displayed. Results may be stored in the device itself, in the user's smartphone, in the cloud or elsewhere, or combinations of these. Results may be displayed on the device, via a smartphone app, or via software accessible via a web browser.

The data from multiple users may be combined to generate maps of regional areas that may indicate a rise in coughing. These data could be used to better predict, for example, when allergenic-pollen laden air from out-of-town will blow into a particular user's neighborhood. The data from a cough/sneeze monitoring device may also be used to identify when the flu season starts and whether it is becoming a pandemic in certain areas.

The pollen sampling device, allergic reaction monitoring device, or both has the ability to keep a time stamp and provide the results to a doctor for further medical diagnosis, analysis, and treatment of the person's respiratory ailment.

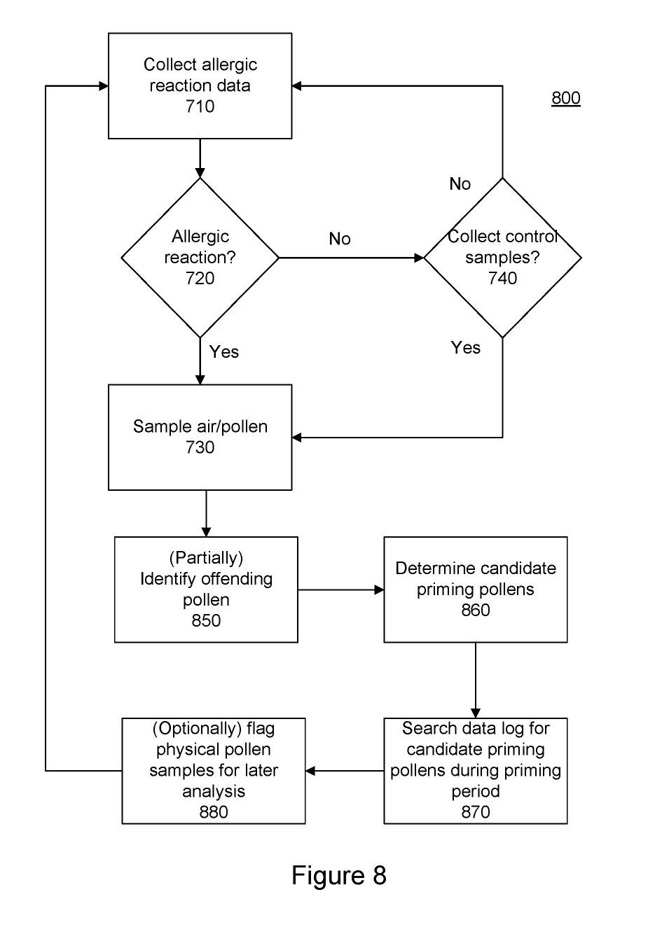

FIG. 8 shows a flow 800 of the system according to another specific embodiment. This process includes the same steps 710, 720, 730 and 740 of flow 700 of FIG. 7. The flow shown in FIG. 8 further includes steps 850, 860, 870 and 880.

In a step 850, data collected from sampled pollen or other allergenic particles is analyzed to determine, or partially determine, what triggered the user's allergic reaction. Based on the type of allergen, or possible types of allergens, determined to have triggered the allergic reaction, a list of candidate priming allergens is constructed in a step 860. This list of candidate priming allergens includes all the triggering or aggravating allergen(s) of step 850 as well as any related cross-reacting allergens. For example, if olive-tree pollen is determined to be the aggravating allergen in step 850, then the list of candidate priming allergens of step 860 would not only include olive-tree pollen, but also privet-tree pollen which is known to cross react with olive-tree pollen.

Associated with an allergic reaction of step 720 is an associated priming period. For example, if the allergic reaction of step 720 occurs on Sunday, one might define a priming period as the previous Monday through Saturday. Generally, it is preferable to use the latest medical knowledge regarding the most appropriate estimate of the duration of the priming period. See earlier discussion related to FIG. 28.

In step 870, past data logs within the priming periods corresponding to allergic reactions are retrieved, reviewed and exposures of the user to any of the allergens in a list of candidate priming allergens is identified.

In some cases a unique aggravating allergen will be determined in step 850 and also a unique priming allergen will be determined in step 870. Such information may be useful to the user and the user's doctor.

In some cases the type of aggravating allergen is not uniquely identified in step 850, however only one unique priming allergen is identified in step 870. Sometimes in such cases, knowledge of the priming allergen will provide the extra clue needed to then uniquely identify the aggravating allergen of step 850. Similarly, a narrowed list of candidate priming allergens from step 870 may reduce the number of possible allergens of step 850, if not uniquely identify the aggravating allergen. Note that in these scenarios, the priming effect is used to better identify the aggravating allergen.

In cases where either the aggravating or the priming allergen has not been uniquely identified by step 870, optional step 880 may be of interest. In step 880, specific grains of pollen or other allergen that have been captured, e.g., in an adhesive-coated tape of the particle collection device, may be identified as being of interest for further analysis. For example, at a later time after the captured pollen has been retrieved and delivered to a laboratory, a wide range of tests may be performed, such as bio-assays, in order to confidently determine the nature of the flagged particles.

Returning to the science of the priming effect, it is not always a requirement that the priming allergen entered the user's body the same way as the triggering (i.e. aggravating) allergen. For example, if the triggering allergen entered through the nose and airway passages, the priming allergen may have entered the user's body through a different route, for example, in food entering the digestive systems. With this in mind, step 870 may include a search of the user's dietary records as well as pollen exposure.

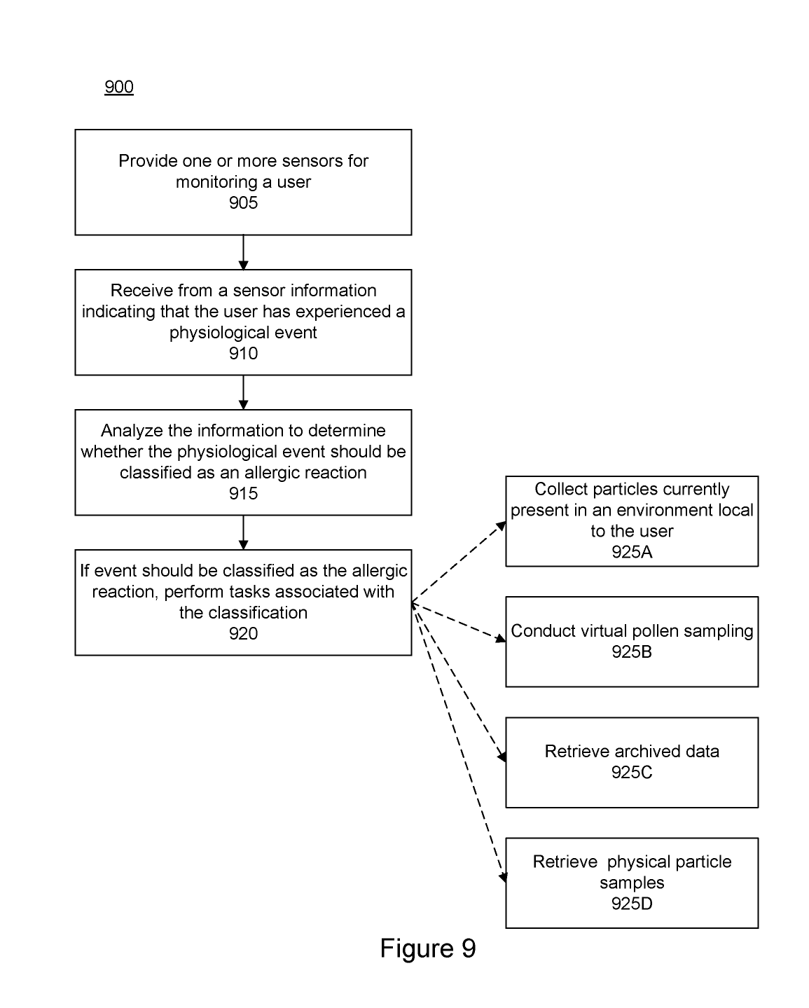

FIG. 9 shows a flow 900 of a process of the system according to a specific embodiment. In a step 905, one or more sensors are provided for monitoring a user for an allergic reaction. As discussed above, the sensors may include a microphone, accelerometer, gyroscope, camera, or any device capable of generating a signal in response to physiological event. Sensors may be worn by the user, attached to the user, deployed in the user's local environment such as in various rooms of the user's house or apartment, or the user's yard, office, or automobile.

In a step 910, the system receives from a sensor information indicating that the user has experienced a physiological event. The information may include, for example, an audio signal generated in response to the user coughing or sneezing, motion data associated with movements the user may have performed voluntarily or involuntarily in response to the coughing or sneezing, images of the user's eyes that may reveal tearing or other irritation or discomfort, or combinations of these.

In a step 915, the system analyzes the received sensor information to determine whether the physiological event should be classified as an allergic reaction. The analysis may include comparing characteristics of a received audio signal against sound characteristics indicative of an allergic reaction (e.g., cough or sneeze), comparing characteristics of received motion data against motion characteristics indicative of the allergic reaction, comparing characteristics of a received image of the user's eye against an image of the eye under an allergic reaction, analyzing images using facial recognition techniques, analyzing the information using artificial intelligence techniques, or combinations of these.

In a step 920, if the system determines that the event should be classified as an allergic reaction, the system performs tasks associated with the classification. The tasks may include, collecting airborne particles currently present in an environment local to the user (step 925A), conducting virtual pollen sampling (step 925B), retrieving archived data (step 925C), retrieving physical particle samples (step 925D), or combinations of these.

Collecting airborne particles may include issuing a request to the local particle collection device to conduct a sampling. The request may be issued from the monitoring device to the particle collection device. The request may be issued from the analysis server to the collection device. The request may be generated internally from within the collection device such as in implementations where the collection device includes a sensor (e.g., microphone). The collection device receives the request and conducts a sampling of the ambient air.

In some cases, the collection device may be set to periodically sample the ambient air to collect control samples. If the collection device receives the collection request while the collection device is between collection periods, the collection device can immediately initiate a sampling. Alternatively, if the collection device receives the collection request during a scheduled collection period, the collection device may temporarily extend the duration of the collection period. Extending the duration of the collection period helps to ensure that the particle responsible for contributing to the user's allergic reaction is collected. The duration may be extended by any amount of time. For example, the duration may be extended by 1, 2, 3, 4, 5, 6, 7, 8, 9, 10, or more than 10 minutes, or less than 1 minute.

Conducting a virtual pollen sampling may include logging the allergic reaction and tagging the allergic reaction with metadata including, for example, a timestamp, location stamp, local weather data (e.g., temperature or humidity), other metadata, or combinations of these. The information may be transmitted to the remote cloud server and cross-referenced or correlated with blooming seasons of various sources of pollen.

Virtual pollen sampling may include conducting a review of pollen identified via one or more other pollen collection devices that may be near a particular user's local area. For example, in some case the particular user who suffers an allergic reaction may not have their own pollen collection device or their collection device may be malfunctioning. While the particular user might not have their own pollen collection device, the particular user's neighbor might have a pollen collection device, there may be a public pollen collection device near the user (e.g., located across the street from the user's home), or both. In a specific embodiment, pollen information gathered from other collection devices within a certain radius of the particular user may be accessed and analyzed to narrow the potential allergens responsible for the particular user's allergic reaction. The particles (e.g., pollens) collected from these other collection devices may be representative of the particles currently present in the user's local environment. The radius can be configurable such as by the user or administrator of the system. The radius may range from about 5 meters to about 1,000 meters. This includes, for example, 10, 50, 100, 250, 450, 650, 850, 999 meters, more than 999 meters, or less than 10 meters.

Instead or additionally, a request may be transmitted to a drone. The drone may be an unmanned aerial vehicle or self-driving automobile. The drone may include a camera so that it can acquire pictures of the user's local environment. The pictures can be analyzed to identify vegetation present in the user's local environment. The identified vegetation can be cross-referenced with blooming seasons associated with the vegetation to narrow the types of pollen that may be responsible for the user's allergic reaction. The drone may include a pollen collection mechanism to collect pollen local to the user.

Retrieving archived data may include designating a set of time periods before the allergic reaction as being an aggravation period, pre-aggravation period, and priming period; accessing images of pollen collected during the aggravation, pre-aggravation, and priming periods; identifying the pollen; and analyzing the periods in conjunction or with respect to the identified pollen. As discussed, for example, in the description accompanying FIG. 28, the time period before an allergic reaction occurs and the user's exposure to pollen types during particular times within that time period can help guide determinations of the pollen type responsible for the user's allergic reaction.

There can be overlaps among the aggravation, pre-aggravation, and priming periods. An ending time of a period may overlap with the starting time of a subsequent period. The ending time of the period may be after the starting time of the subsequent period. The starting time of the subsequent period may be before the ending time of a previous period. Having overlapping periods help to address and account for the many different variables that can affect an individual user's immune system and the immune system's subsequent response in relation to the aggravation, pre-aggravation, and priming periods.

Retrieving physical particle samples may include accessing a particle collection cartridge having a tape media containing particles collected during a time period before the allergic reaction; designating portions of the time period as being aggravating, pre-aggravating, and priming periods; identifying pollens collected during the aggravating, pre-aggravating, and priming periods; and analyzing the periods in conjunction or with respect to the identified pollen.

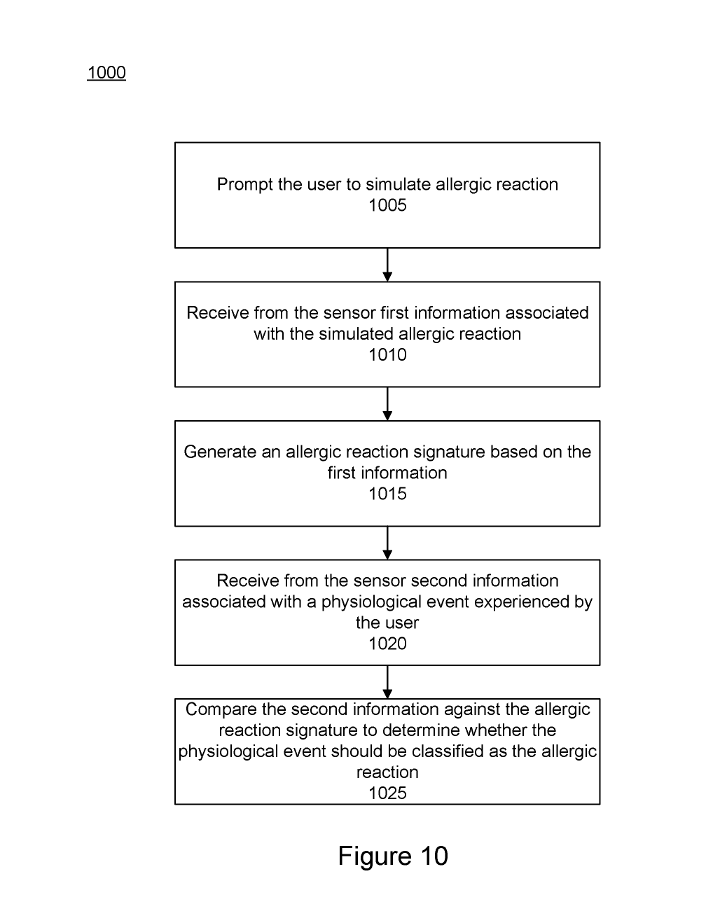

FIG. 10 shows a flow 1000 of a process for generating an allergic reaction signature according to another specific embodiment. In a step 1005, the system prompts the user to simulate an allergic reaction. At least one sensor detects a physiological event associated with the simulated allergic reaction.

In a step 1010, first information associated with the simulated allergic reaction is received from the at least one sensor. In a step 1015, the system generates and stores an allergic reaction signature based on the first information.

In a step 1020, second information associated with a physiological event experienced by the user is received from a sensor. In a step 1025, the system compares the second information against the allergic reaction signature to determine whether the physiological event should be classified as an allergic reaction. As discussed above, an allergic reaction signature may be based on, for example, sounds of coughing, sneezing, or both. An allergic reaction signature may be based on activity or movements associated with coughing, sneezing, or both.

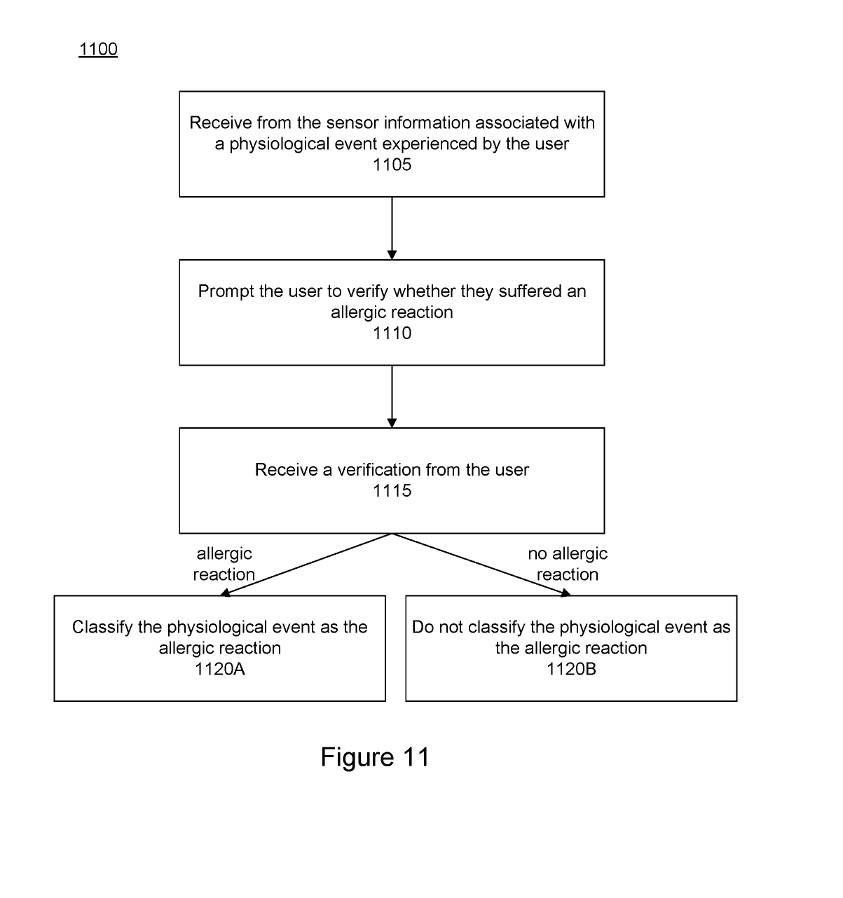

FIG. 11 shows a flow 1100 of a process for prompting a user to verify whether they suffered an allergic reaction. In a step 1105, information that may be associated with a physiological event experienced by the user is received from a sensor. For example, a microphone may generate an acoustic signal in response to detecting a sound. An accelerometer may generate an activity signal in response to detecting movement. These signals can be flagged as a candidate or potential allergic reaction.

In a step 1110, the system prompts the user to verify whether they have just suffered an allergic reaction. For example, the system may display or cause to be displayed on the electronic smartphone screen of the user the message “Have you just suffered an allergic reaction?” and a set of “Yes” and “No” buttons that the user can select. The system prompts the user to confirm or deny that they genuinely coughed or sneezed.

In a specific embodiment, the system stores a default allergic reaction signature. Prior to prompting the user, the received information is compared against the default allergic reaction signature. The user is not prompted until the received information passes this initial threshold test. This helps to ensure that other ambient noise detected by the sensor such as a barking dog does not result in pestering the user with verification prompts.

In a step 1115, a verification is received from the user. For example, the user may respond by clicking the “yes” button to verify that they have suffered an allergic reaction. Alternatively, the user may respond by clicking the “no” button to verify that they have not suffered an allergic reaction.

In a step 1120A, if the user verifies that they have suffered an allergic reaction, the system classifies the physiological event as an allergic reaction. Alternatively, if the user indicates that they have not suffered an allergic reaction, the system does not classify the physiological event as an allergic reaction. In a specific embodiment, the user's response to the verification is used by the system to adjust or replace the default allergic reaction signature. For example, if the user's response is that the event was not an allergic reaction, the system can adjust the default allergic reaction signature based on, for example, an audio signal associated with the event so that the next time a similar audio signal is received the system will not prompt the user with a verification.

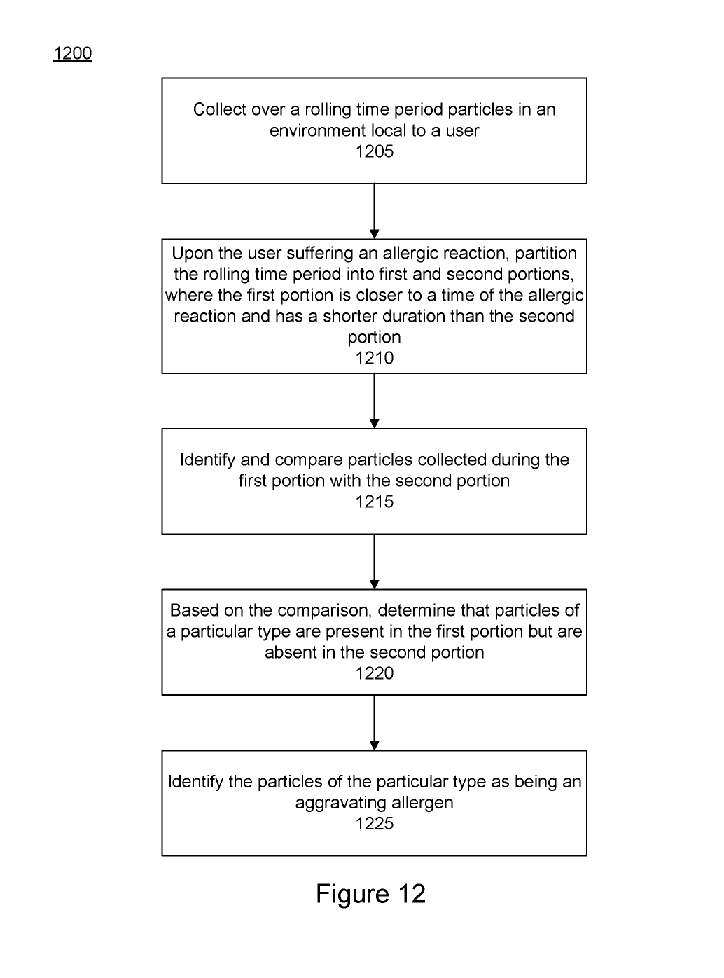

FIG. 12 shows a flow 1200 of a process for identifying an aggravating allergen according to a specific embodiment. In a step 1205, the system collects over a rolling time period particles in an environment local to the user. The rolling time period can be configured by the user or administrator to be of any duration. For example, the rolling time period can be 1, 2, 3, 4, 5, 6, 7, 8, 9, 10, or more than 10 days. The rolling time period can be less than 1 day (e.g., 9 hours).

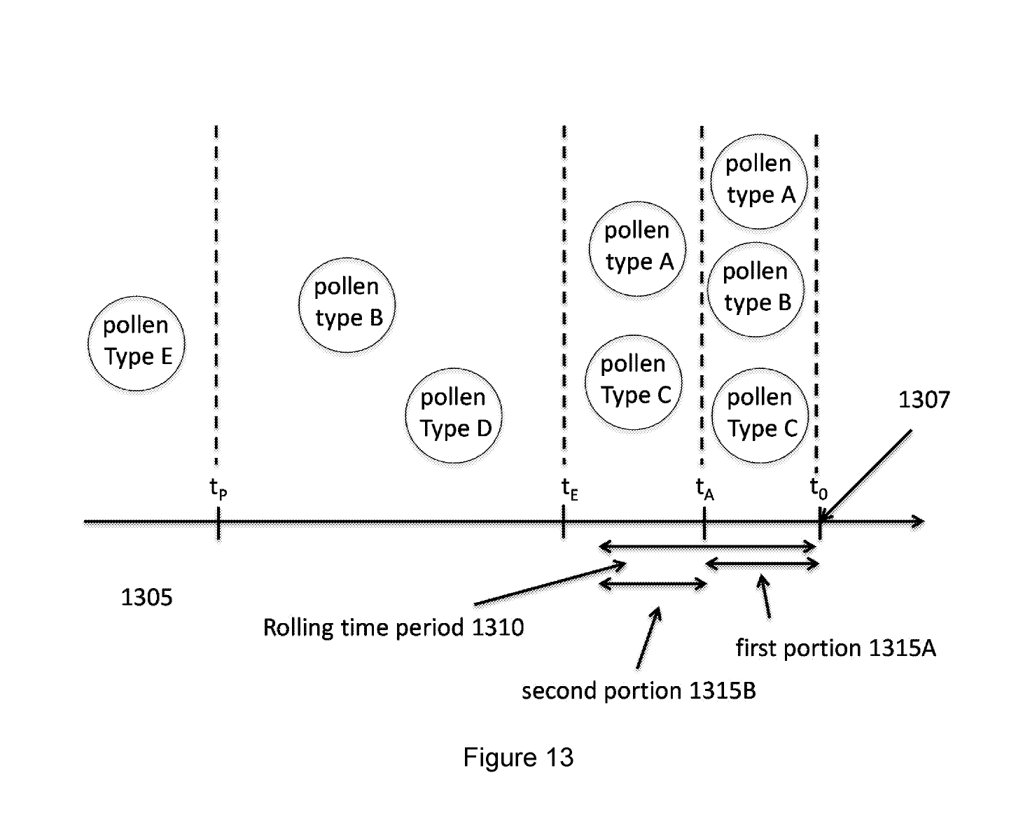

In a step 1210, upon a determination that the user has suffered an allergic reaction, the rolling time period is partitioned into first and second portions, and perhaps additional portions. The first portion is closer to a time of the allergic reaction and has a shorter duration than the second portion. For example, FIG. 13 shows a timeline 1305 that includes a priming-period start time t_{P}, a priming-period end time t_{E}, and aggravation-period start time t_{A} and an allergic reaction start time t₀. The user has suffered an allergic reaction 1307.

Consider, as an example, that a rolling time period 1310 has been set to some time after the priming-period end time t_{E} but well before the aggravation-period start time t_{A}. For clarity of presentation, the time axis in FIG. 13 is distorted. If drawn to scale, the first portion 1315 would be too compressed to have room on the page for the aggravation period pollen types.

The rolling time period is partitioned into a first portion 1315A and a second portion 1315B. Optionally there may be a gap in time between the first portion 1315A and the second portion 1315B. As shown in the example of FIG. 13, the first portion is closer to a time of the allergic reaction and preferably has a shorter duration than the second portion. The first portion 1315A may extend from the aggravation-period start time of t_{A} to the allergic reaction time t₀. The second portion may commence at or after the priming-period end time t_{E} and conclude at or before the aggravation-period start time t_{A}. In other words, the second portion 1315B is preferably equal to or is a sub-interval of the pre-aggravation period between times t_{E} and t_{A}.

Referring back now to FIG. 12, in a step 1215, the system identifies and compares and correlates particles collected during the first portion of the rolling time period and particles collected during the second portion of the rolling time period.

In a step 1220, based on the comparison, a determination is made that particles of a particular type are present in the first portion, but are absent in the second portion. In a step 1225, the particles of the particular type are identified as being an aggravating allergen.

For example, referring now to FIG. 13, pollen types A, B, and C were found to be present during the first portion of the rolling time period. Pollen types A and C were found to be present during the second portion of the rolling time period. The two collections are compared (step 1220). The comparison indicates that pollen types A and C are present in both the first and second portions. Pollen type B, however, is present in the first portion, but is absent from the second portion. A determination may then be made that pollen type B is the aggravating pollen because it was present at the time of the allergic reaction and (unlike the other types of pollen) was not present in the preceding second portion of the time period during which there was no allergic reaction.

In other words, in a specific embodiment, prior to the user's allergic reaction, ambient air is being periodically sampled and pollen type A as well as pollen type C are observed, but not pollen type B. When the user does have an allergic reaction, pollen types A, B and C are all observed. A conclusion can then be made that the aggravating pollen is of type B, as its arrival on the scene correlated to the onset of an allergic reaction.

Optionally, in step 1225, the user may be asked questions regarding medication usage. If, for example, the user had taken an anti-histamine medication whose effects may plausibly have faded at the aggravation-period start time 2830, pollen types A, B and C may all still be candidate aggravating pollens as the lack of allergic reaction during second portion 1315B may have been due to medication rather than lack of exposure.

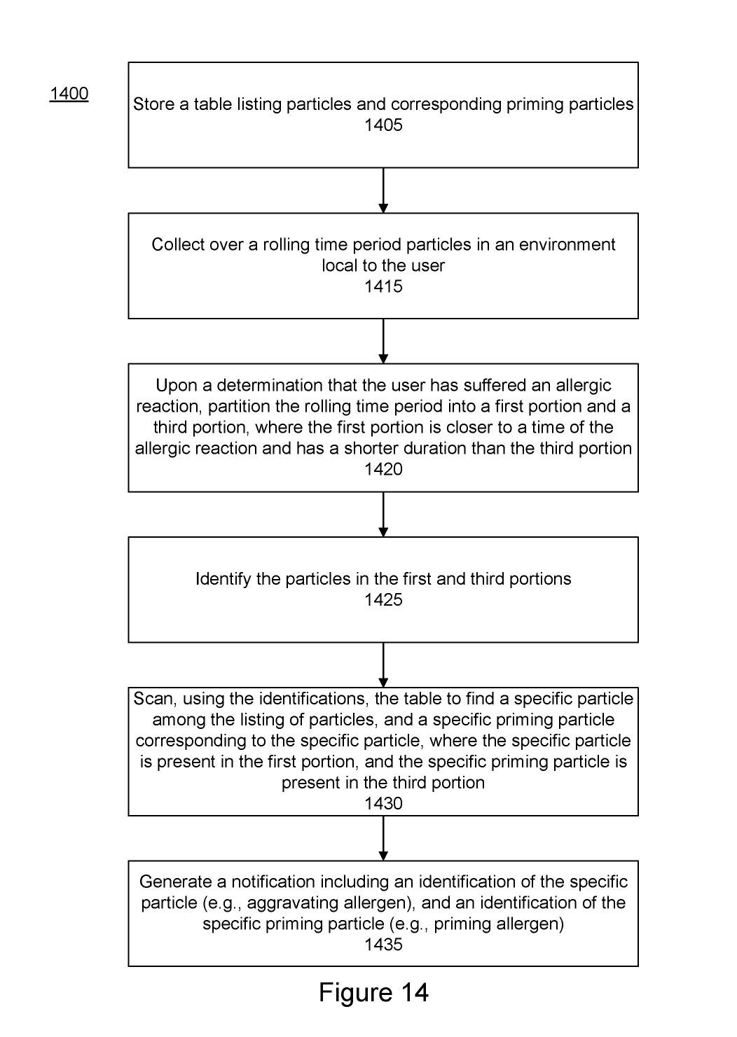

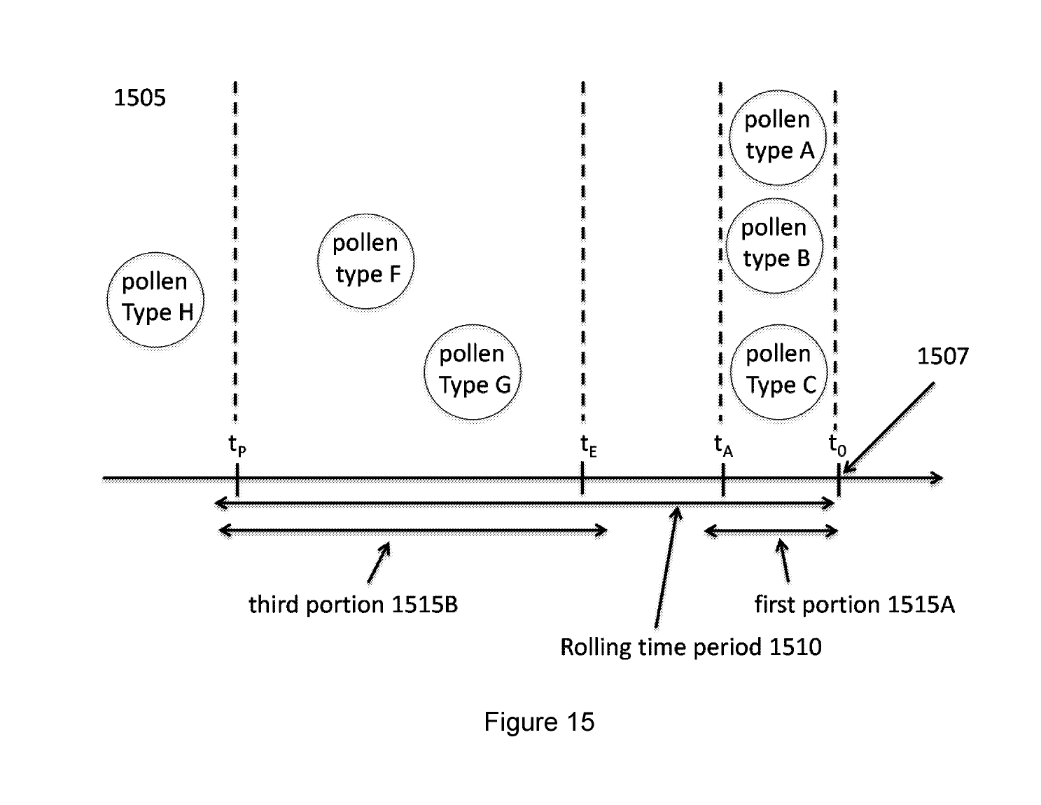

An allergic reaction to pollen type B implies that during the aggravation period the user's immune system was primed for pollen type B. In the scenario shown in FIG. 13, the immune system was primed by an earlier exposure to pollen type B during the priming period. The user may well have been exposed to other pollens during the priming period, such as pollen type D. Any pollens, such as pollen type E, present before the priming period are too early to have any effect on user symptoms at allergic reaction time t₀. Optionally, process 1200 may be extended to take advantage of the presence of pollen type B during the priming period to confirm the diagnosis that pollen type B is the aggravating pollen. The use of data from the priming period will be considered in more detail below in connection with FIGS. 14 and 15.

A duration of the first portion of the rolling time period may range from about 1 minute to about 30 minutes. This includes, for example, 2, 3, 4, 5, 6, 7, 8, 9, 10, 11, 12, 13, 14, 15, 20, 25, or more than 25 minutes. The duration may be greater than 30 minutes. The duration may be less than 1 minute. The first portion 1315A is preferably equal to, or a superset of the aggravation period between times t_{A} and to.

A duration of the second portion of the rolling time period may range from about 30 minutes to about two days. This includes, for example, 30 minutes, 1, 2, 3, 6, 12 hours, 1, or 2 days. The duration may be greater than 2 days. The duration may be less than 30 minutes. The durations can be configurable such as by a user to administrator of the system. The second portion is preferably equal to or a subset of the pre-aggravation period between t_{E} and t_{A}.

FIG. 13 illustrates an absence of pollen type B in the second portion 1315B. This may correspond to a case where absolutely no pollen type B particles are detected during second portion 1315B. Alternately, FIG. 13 may also represent a case where pollen type B particles are detected but at a level too low to aggravate a noticeable allergic reaction. Interestingly, medical science suggests that it is possible for a low level of exposure to be insufficient to aggravate allergic reaction systems and yet be sufficient to keep the user's immune system primed to the allergen. In the medical literature, this situation may be referred to as “minimum persistent inflammation.” Thus, in a specific embodiment, “aggravation” as described herein may have or be associated with a higher exposure threshold than “priming.”

In a specific embodiment, a configuration setting of the system includes a threshold pollen count. A pollen count is the measurement of the number of grains of pollen in a cubic meter of air. Instead or additionally, there can be another or different threshold parameter that indicates a required threshold level of pollen concentration that must be met or exceeded for the system to make a determination that a particular type of pollen is present. Requiring that there be at least a threshold concentration level of pollen helps to indicate that a user was actually exposed to the pollen and was exposed to a sufficient concentration of the pollen for the user to be affected.

In a specific embodiment, a component of the system, such as the local particle collection device, measures a concentration of a particular type of detected pollen. The concentration is compared against the stored threshold concentration level. If the concentration is above the threshold concentration level, the system determines that the particular pollen type is present. If the concentration is below the threshold concentration level, the system determines that the particular pollen type is absent.

Each of the priming, pre-aggravation, and aggravation periods may have the same or different threshold concentration level requirements. For example, the aggravation period may be associated with a first threshold concentration level. The pre-aggravation period may be associated with a second threshold concentration level. The priming period may be associated with a third threshold concentration level. A concentration level of a particular type of pollen detected during the aggravation period may be compared against the first threshold concentration level. If the concentration level exceeds the first threshold concentration level, a determination is made that the particular pollen type is present during the aggravation period. If the concentration level does not exceed the first threshold concentration level, a determination is made that the particular pollen type is not present during the aggravation period.

A concentration level of a particular type of pollen detected during the pre-aggravation period may be compared against the second threshold concentration level. If the concentration level exceeds the second threshold concentration level, a determination is made that the particular pollen type is present during the pre-aggravation period. If the concentration level does not exceed the second threshold concentration level, a determination is made that the particular pollen type is not present during the pre-aggravation period.

A concentration level of a particular type of pollen detected during the priming period may be compared against the third threshold concentration level. If the concentration level exceeds the third threshold concentration level, a determination is made that the particular pollen type is present during the priming period. If the concentration level does not exceed the third threshold concentration level, a determination is made that the particular pollen type is not present during the priming period. The first threshold concentration level may be the same as the second threshold concentration level, the third threshold concentration level, or both. The first threshold concentration level may be different from the second threshold concentration level, the third threshold concentration level, or both. The second threshold concentration level may be the same as or different from the third threshold concentration level.

The threshold concentration levels for different pollen types can be the same or different. For example, in some cases a particular pollen type may need to be present in higher concentrations than another pollen type in order for a user to be affected by the particular pollen type as compared to the other pollen type. A first pollen type may be associated with a first threshold concentration level. A second pollen type, different from the first pollen type, may be associated with a second threshold concentration level. The second threshold concentration level may be the same as or different form the first concentration level.

Different users may have differing levels of pollen sensitivity. A component of the system, such as the local pollen collection device, may be configured or customized accordingly for each different user. For example, a first local pollen collection device for a first user may be associated with a first threshold concentration level for pollen. A second local pollen collection device for a second user, different from the first user, may be associated with a second threshold concentration level for pollen. The second threshold concentration level may be the same as or different from the first threshold concentration level.

FIG. 14 shows a flow 1400 of a process for identifying aggravating and priming allergens or particles according to a specific embodiment. In a step 1405, the system stores a table listing particles (or aggravating allergens) and corresponding priming particles (or priming allergens). For example, the table may be stored by the analysis server or particle collection device. Table D below shows an example of information that may be stored by the system.

TABLE D

| Aggravating Pollen | Corresponding Priming Pollens |
|---|---|
| Pollen Type A | Pollen Type A |
| Pollen Type B | Pollen Type B, G |
| Pollen Type C | Pollen Type C, H |
| . . . | . . . |

Table D above cross-references a specific pollen type with a related corresponding pollen type that is known to be a priming pollen of the specific pollen type. As discussed, the priming pollen may be the specific pollen type itself. However, due to cross-reactivity, in addition to itself, an aggravating pollen may be associated with other additional priming pollens. The table indicates that a user who has an allergic reaction to pollen type A would have initially been introduced to pollen type A during a priming period. A user who has an allergic reaction to pollen type B would have initially been introduced to pollen type B, G, or both during a priming period. A user who has an allergic reaction to pollen type C would have initially been introduced to pollen type C, H, or both, and so forth. The relationship between an aggravating and priming pollen can be one-to-one, one-to-many, many-to-one, or many-to-many.

In a step 1415, the system collects over a rolling time period particles (e.g., pollen) in an environment local to the user.

In a step 1420, upon a determination that the user has suffered an allergic reaction, the rolling time period is partitioned into a first portion, preferably including the aggravation period from time t_{A} to time t₀, and a third portion preferably including the priming period from t_{P} to t_{E}. Preferably there is a time gap between the first and third portions. As the priming period is much longer in duration than the aggravation period, the third portion is preferably much longer than the first portion. Also, as the priming period occurs before the aggravation period, the third portion corresponds to earlier times than the first portion.

In a step 1425, the particles collected in the first and third portions of the rolling time period are identified.

In a step 1430, the system scans, using the identifications, the table to find a specific particle among the listing of particles, and a specific priming particle corresponding to the specific particle, where the specific particle is present in the first portion related to the aggravation period, and the specific priming particle is present in the third portion related to the priming period.

For example, FIG. 15 shows a timeline 1505 that includes a priming-period start time t_{P}, a priming-period end time t_{E}, and aggravation-period start time t_{A} and an allergic reaction start time t₀. The user has suffered an allergic reaction 1507. Consider, as an example, that a rolling time period 1510 has been set to start on or before the priming-period start time t_{P} and end at time t₀ of allergic reaction 1507.

The rolling time period is partitioned into a first portion 1515A, preferably including the aggravation period between t_{A} and to, and a third portion 1515B, preferably including the priming period between t_{P} and t_{E}. As shown in the example of FIG. 15, the first portion is closer to a time of the allergic reaction and has a shorter duration than the third portion.

Referring back now to FIG. 14, in a step 1425, the system identifies the particles collected during the first portion associated with the aggravation period and the third portion associated with the priming period.

In a step 1430, the system scans, using the identifications, the table to find a specific particle among the listing of particles, and a specific priming particle corresponding to the specific particle, where the specific particle is present in the first portion associated with the aggravation period, and the specific priming particle is present in the third portion associated with the priming period.

For example, referring now to FIG. 15, pollen types F and G were found to be present during the third portion associated with the priming period. Pollen types A, B and C were found to be present during the first portion associated with the aggravation period.

The listing of pollen types identified from the priming time period can then be searched for candidate priming pollen types A, B, C, G, and H. In this example, the priming time period included pollen types F and G. Candidate priming pollen types A, B, C and H can be eliminated from consideration because they were not present during the third portion including the priming period.

Similarly, pollen types A and C can be eliminated from consideration as aggravating pollen types because their corresponding priming pollen types (pollen types A and C and H, respectively) were not present during the third portion including the priming period. Candidate priming pollen type G (corresponding to aggravating pollen type B), however, was present during the third portion including the priming period and can thus be identified as the priming pollen.

In a step 1435, a notification is then generated that identifies the specific particle (e.g., aggravating pollen type B) and corresponding priming particle (e.g., priming pollen type G).

In further embodiments, conclusions derived from the processes such as process 1200 and process 1400 are not definitive but rather probabilistic and provide inputs to other processes that may use fuzzy logic or other algorithms to combine results with other information. For example, if in a given geographical area, multiple users are suffering similar allergic symptoms, and each individually have multiple candidate aggravating pollen types and multiple candidate priming pollen types, but there is only one allergen pollen that is likely to explain the allergic reaction of all the users, and there is only one likely candidate priming pollen consistent with all the users, then algorithms considering probabilistic results from all the users may reach reliable conclusions regarding offending allergens.

In a specific embodiment, a rolling time period is between one week and one month prior to the allergic reaction of the user. The system allows the timing of the first and third portions to be adjustable such as by a user or administrator of the system. The adjustability allows the system to be fine-tuned and customized for particular users, local environments, and applications.

In some applications a timing of the first portion of the embodiments of FIG. 15 may be similar to the timing of the first portion of the embodiments of FIG. 13.

In a specific embodiment, particles collected in the absence of an allergic reaction are archived and stored by the collection device. Physical samples of pollen may be captured and retained for possible future retrieval. In a specific embodiment, captured particles are not analyzed or not imaged until the allergic reaction occurs. The occurrence of allergic reaction can trigger the retrieval of physical samples of previously collected pollen. Not analyzing the particles until an allergic reaction occurs can help to conserve system resources. In some cases, there may be many rolling priming time periods that will have elapsed before an allergic reaction occurs. Analyses performed on prior priming time periods that have elapsed may not be relevant. In this specific embodiment, a method includes collecting over a plurality of rolling time periods particles in a local environment, and upon determining that the user has suffered an allergic reaction, accessing particles collected during a current rolling time period for analysis, where particles collected during rolling time periods before the current rolling time period are not analyzed. The particles collected during the elapsed rolling time periods before the current rolling time period may be removed or cleaned from the collection device.

In another specific embodiment, particles collected in the absence of an allergic reaction are analyzed (e.g., imaged) even though an allergic reaction may have yet to occur. Images and other data of the collected particles may be stored and archived so that the data can be later retrieved to identify the priming allergen when an allergic reaction occurs. The occurrence of allergic reaction can trigger the retrieval of images or other data of previously collected pollen.

Once images of the particles have been captured, the physical particles may or may not be removed or cleaned from the collection device. Removing or cleaning the particles from the collection device helps to conserve physical storage space. Maintaining the physical particles, however, allows for further analysis such as later bioassays if desired. Particle images associated with elapsed rolling time periods may be deleted. The system provides options and flexibility to allow users or administrators to decide whether or not the physical particle samples, particle images, or both should be archived and maintained or removed.

In a specific embodiment, a method includes receiving a first duration associated with an aggravating period, a second duration associated with a pre-aggravating period, and a third duration associated with a priming period; collecting, over a period of time, pollen present in a local environment of a user, each collected pollen being associated with a time and date indicating when the pollen was collected; determining that the user has suffered an allergic reaction; partitioning the period of time over which the pollen present in the local environment of the user was collected into the aggravating period, the pre-aggravating period, and the priming period; identifying pollen collected during the aggravating, pre-aggravating, and priming periods; examining the pollen identifications relative to the aggravating, pre-aggravating, and priming periods to identify an aggravating pollen for the allergic reaction; and generating a report comprising an identification of the aggravating pollen, wherein the aggravating pollen is present during the aggravating period, and is absent during the pre-aggravating period, wherein the aggravating pollen or a pollen identified as having cross-reactivity with the aggravating pollen is present during the priming period, wherein the aggravating period is closer to time of the allergic reaction than the pre-aggravating and priming periods, wherein the duration of the aggravating period and the pre-aggravation period are less than the duration of the priming period, and wherein the pre-aggravating period is between the priming and aggravating periods.

A deployment of the system may include providing local particle collection devices to any number of different users. In a specific embodiment, the particle collection devices can be configured remotely from a central management station. The particle collection device can communicate with the central management station over a network such as the Internet. The particle collection device may receive commands and instructions from the central management station over the network such as the Internet.

A particle collection device may be configured independently of another particle collection device. For example, a first particle collection device may receive a first set of durations associated with aggravating, pre-aggravating, and priming periods. A second particle collection device may receive a second set of durations associated with aggravating, pre-aggravating, and priming periods, where at least one duration in the second set of durations is different from a corresponding duration in the first set of durations.

The method may further include identifying a priming pollen, wherein the priming pollen is present during the priming period and is of a same pollen type as the aggravating pollen. The method may further include identifying a priming pollen, wherein the priming pollen is present during the priming period, is of a different pollen type than the aggravating pollen, and comprises a cross-reactivity with the aggravating pollen.

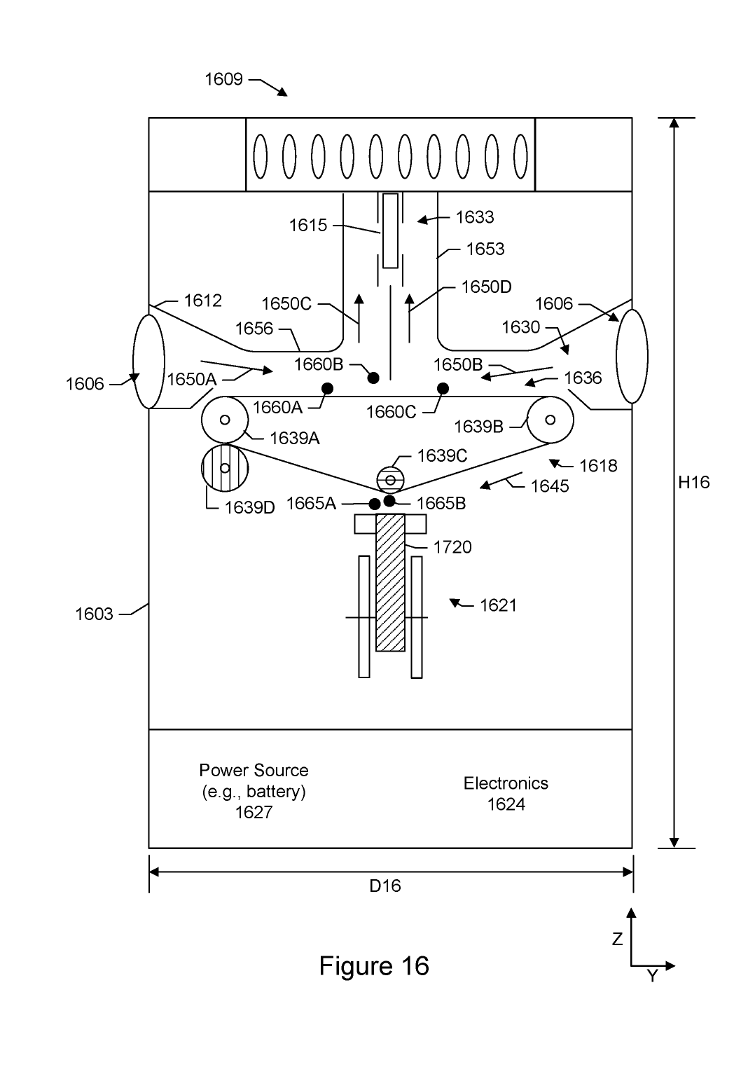

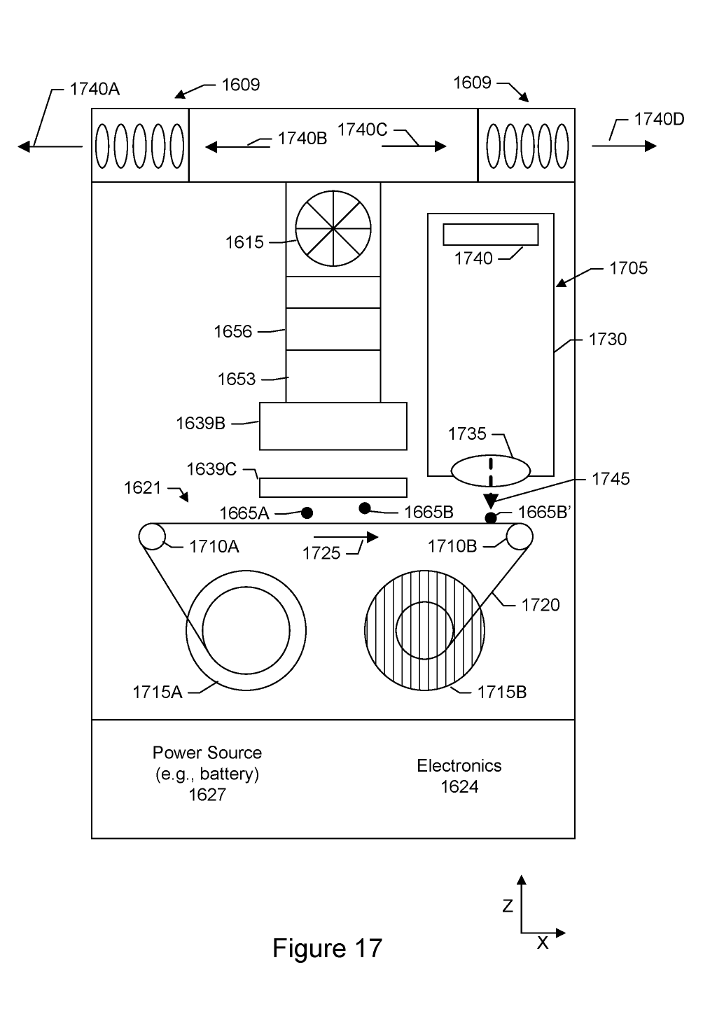

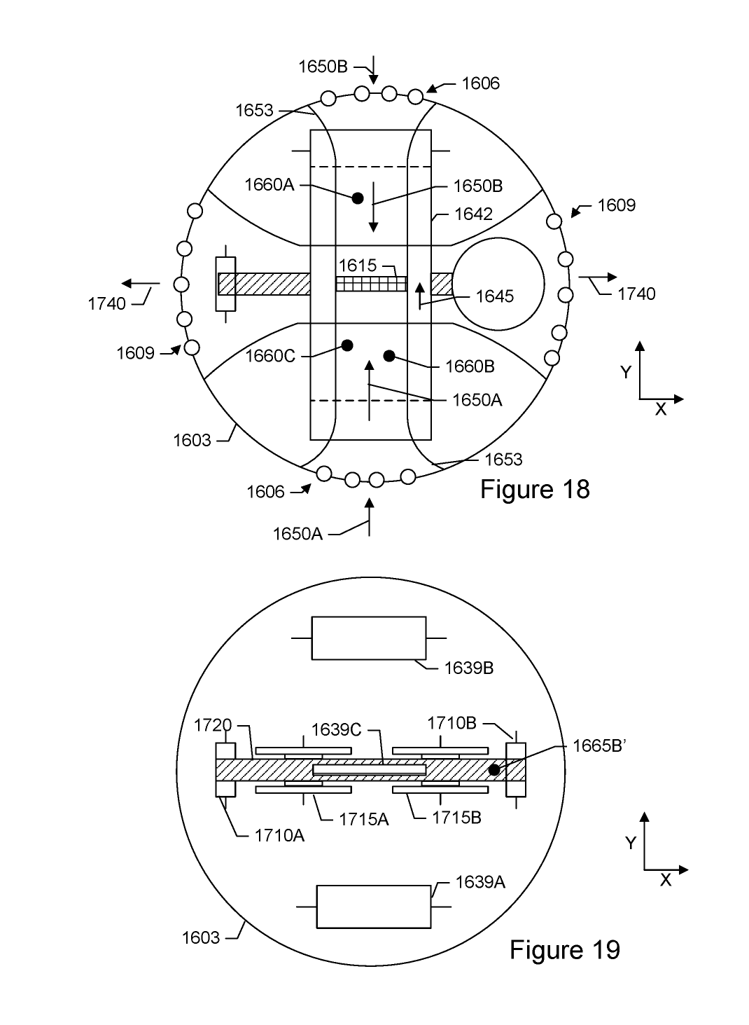

FIG. 16-19 show various views of a particle or pollen collection device 1600 according to a specific embodiment. The pollen collection device samples ambient air, captures pollen on an adhesive-coated tape, images captured pollen with a camera lens and camera sensor, and archives adhesive coated tape with captured pollen on a take-up reel. Camera-image data and the results of its analysis may be stored or logged for later use. Likewise, physical samples of pollen and other particles may be stored or archived for possible later retrieval. FIG. 16 shows a side view of the collection device in a Y-Z plane. FIG. 17 shows a side view of the collection device in an X-Z plane. FIG. 18 shows a plan view of the collection device. FIG. 19 shows another plan view of the collection device.

Referring now to FIG. 16, this particle collection device includes a cylindrical enclosure, cabinet, or housing 1603 having a set of intake vent holes 1606 and a set of outtake or exhaust vent holes 1609. The intake vents are located on a side surface of the enclosure between a top end of the enclosure and a bottom end of the enclosure, opposite the top end. The intake vents are positioned closer to the top of the enclosure than the bottom. The outtake vents are at the top of the enclosure.

Internal components include a duct 1612 connected between the intake and outtake vents, a blower 1615 positioned inside the duct, a first conveyor assembly 1618 below the duct, a second conveyor assembly 1621 below the first conveyor assembly, an optical microscope 1705 (FIG. 17), electronics 1624 (e.g., processor or network interface card), and a power source (e.g., battery) 1627. The power source and electronics are housed at the bottom of the enclosure. The power source supplies power to the blower, conveyor assemblies, optical microscope, and other electrical components of the collection device.

The duct includes a horizontal segment 1630 and a vertical segment 1633. In the example shown in FIG. 16, a bottom end of the vertical segment is connected to a middle portion of the horizontal segment. The vertical segment extends along a central or longitudinal axis of the enclosure. The horizontal segment is orthogonal to the vertical segment. The intake vents open into the horizontal segment of the duct. The outtake vents are at a top of the vertical segment of the duct. A bottom portion of the horizontal segment includes an opening 1636 between opposite intake vents.

First conveyor assembly 1618 includes rollers 1639A, B, C, and D, and a non-stick tape 1642 (see FIG. 18). A roller may be referred to as a pulley or drum. The non-stick tape passes around rollers 1639A, B, and C and above roller 1639D. Roller 1639D is controlled by a stepper motor (indicated the figure by a pattern of vertical lines) and is coated with a sticky adhesive. Via roller 1639D, the stepper motor controls the motion of the non-stick tape so that it moves in a direction as indicated by an arrow 1645. A portion of the non-stick tape is exposed through opening 1636. Roller 1639C is provided with a vibrator or mechanism to vibrate at acoustic or ultrasonic frequencies (indicated in the figure by a pattern of horizontal lines).

Second conveyor assembly 1621 is below the first conveyor assembly and is oriented orthogonally to the first conveyor assembly. Second conveyor assembly 1621 (as shown in FIG. 17) includes rollers 1710A and B, reels 1715A and B, and an adhesive coated tape 1720. The adhesive coated tape is supplied by reel 1715A, passes around or is guided by rollers 1710A and B, and is collected by reel 1715B. A motion 1725 of the adhesive coated tape is driven via take-up reel 1715B by a second stepper motor as indicated in the figure by a pattern of vertical lines.

The optical microscope includes an optical column 1730 with objective lens array 1735 and a camera-image sensor 1740.

Referring now to FIG. 16, blower 1615 drives a flow of air into the intake vents and out the outtake vents. More specifically, arrows 1650A-D indicate the flow of sampled ambient air, perhaps containing pollen and other allergenic substances, into the device via the intake vent holes. Vertical walls 1653 (see FIG. 18) and horizontal ceiling 1656 of the duct help channel the incoming air in desired directions. The airflow is driven by the blower. The blower also drives downstream airflow indicated by arrows 1740A-D (FIG. 17). Air exits the device via the outtake vent holes.

The non-stick tape may include a loop of Teflon™ or other material generally regarded as a non-stick material and completes the boundaries for the incoming air flow. The tape forms a loop and may be referred to as a non-stick tape loop. The tape may be, for example, a polymer tape or include a polymer material. Other appropriate materials may instead or additionally be used. Despite use of a tape material generally regarded as non-stick, very small particles such as pollen grains will stick to the surface of the non-stick tape loop as a result of Van der Waals forces. In alternate embodiments, the non-stick tape need not be a loop, but rather can be tape supplied reel to reel for one-time use. In other specific embodiments, the non-stick tape may be cleaned after use so that it can be reused one or more times.

As shown in the example of FIG. 16, at least a portion of the non-stick tape loop is positioned so that it is near airflow 1650A-D. For example, the at least a portion of the non-stick tape may be below the airflow or may be within or at least partially obstruct the airflow. In a specific embodiment, at least a portion of the airflow path passes over opening 1636 of the duct through which at least a portion of the non-stick tape loop is exposed. Due to the force of gravity, pollen grains (e.g., pollen grains 1660A-C) will settle out of the sampled ambient air in the airflow and stick to the surface of the non-stick tape loop that is exposed through the duct opening. When desired, the non-stick tape loop is moved in the direction indicated by arrow 1645.

As discussed above, the loop is supported by rollers 1639A and 1639B. Roller 1639D is controlled by a stepper motor (not shown) and is coated with a sticky adhesive. Via roller 1639D, the stepper motor controls motion 1645 of the non-stick tape loop. Roller 1639C is provided with a mechanism to vibrate at acoustic or ultrasonic frequencies. This results in at least some of the captured pollen grains (e.g., pollen grains 1665A-B) being released under the influence of gravity. Due to its sticky adhesive, roller 1639D will remove any pollen and other particles on the surface of the non-stick tape loop that were not removed by vibration of roller 1639C.

Referring now to FIG. 17, vibration released pollen grains 1665A and 1665B fall and land upon adhesive coated tape 1720. As discussed, the adhesive coated tape is supplied by reel 1715A, is guided by rollers 1710A-B and is collected by reel 1715B. The motion of the adhesive-coated tape is driven via take-up reel 1715B by a second stepper motor (not shown). Motion 1725 of the adhesive-coated tape moves captured pollen grains and other particles, such as pollen grain 1665B′, within a field of view 1745 of the optical microscope.

The optimal or desired field of view will depend on the application. In a specific embodiment, a field of view of width is about 1 millimeter (mm). In a specific embodiment, a width of the field of view is substantially narrower or less than the width of the pollen collection region of non-stick tape 1642, advantageously greatly increasing the concentration of particles in the field of view. For example, a ratio between the field of view of width and the width of the pollen collection region of the non-stick tape may be about 1:2. In other specific embodiments, the ratio may be about 1:1.2, 1:1.4, 1:1.6, 1:1.8, 1:2.2, 1:2.4, 1:2.6, 1:2.8, 1:3, 1:4, 1:5, 1:6, 1:7, 1:8, 1:9, or 1:10.

In a specific embodiment, the exposed horizontal surface area of the non-stick tape loop is about 7 centimeters (cm)×2 cm=14 cm̂2. For smaller pollen particles of diameters of about 20 microns, the pollen settling rate is roughly 1 cm/second, resulting in an air sampling rate of about 14 cm̂2×1 cm/sec=14 cm̂3/sec=840 cm̂3/minute=0.84 liter/minute. This is the case if the blower establishes sufficient airflow so that air reaching the most interior portions of the non-stick tape loop are depositing pollen grains. This is about one order of magnitude less than a typical breathing rate of a resting human. In some applications this may be sufficient. In other applications, it will be desirable to increase the ambient-air sampling rate. Particularly for indoor applications, the relatively fast settling of pollen out of air and the relatively low “wind velocity” indoors may well vary by orders of magnitude from one location to another within a home. Intelligent placement of one or more pollen monitors within a home may well provide a desired order of magnitude increase in effective pollen monitor sensitivity.

In many cases, the two stepper motors may be at rest most of the time. For example, the non-stick tape loop may be at rest for one minute while pollen grains (e.g., pollen grains 1660A-C) accumulate on its surface. After a sufficient such period, the stepper motor driving roller 1639D may be activated and ultrasonic release roller 1639C may be excited for sufficient time to transfer all or at least some of the collected pollen grains onto the adhesive-coated tape (see, e.g., falling grains 1665A-B). Roller 1639D cleans the non-stick polymer loop material in preparation for a subsequent sampling period. For example, the roller may include a brush to clean the non-stick tape of particles. The operation of the stepper motors may be triggered based upon a pre-determined schedule or frequency. Instead or additionally, the operation of the stepper motors may be triggered based on some other event.

As a result, pollen sampled from 840 cm̂3 of ambient air is deposited in a narrow strip below ultrasonic release roller 1639C and onto the adhesive-coated tape. Ideally the width of these deposited pollen grains is no wider than the field of view of camera image sensor 1740 (FIG. 17) so that all pollen collected may be imaged. After the transfer of pollen from the non-stick tape loop to the adhesive-coated tape is complete, then via reel 1715B the second stepper motor may move the collected pollen through the field of view of the microscope system.

In a specific embodiment, an operation of the collection device is triggered upon a determination that the user has suffered an allergic reaction. In this specific embodiment, the collection device receives a request (such as from the analysis server or allergic reaction monitoring device with sensor) to sample the ambient air. Upon receiving the request, the blower is activated. Particles within the airflow suction or vacuum caused by the blower are deposited via gravity through the bottom opening of the duct and onto the waiting non-stick tape of the first conveyor assembly.

Once the period of collection is complete, the non-stick tape may be advanced. When the portion of the non-stick tape having the collected particles reaches roller 1639C, the advancement of the non-stick tape may be paused or slowed while roller 1639C is vibrated to shake the collected particles off and onto the adhesive-coated tape of the second conveyor assembly. Once the shaking is complete, the adhesive-coated tape may be advanced.

When the portion of the adhesive-coated tape having the collected particles reaches or is within the field of view of the camera, the advancement may be paused or slowed so that the camera can capture an image of the particles stuck to the adhesive-coated tape. The captured images are then analyzed to identify the captured particles (e.g., pollen).

A combination of factors including pollen optical properties (e.g., blue to red ratio) and pollen grain size may be evaluated using an algorithm of the system to identify pollen. Pollen grain size can be determined according to scattered light intensity. For example, pollen such as ragweed, Japanese cedar, walnut, and kamogaya may be identified based on their respective blue/red fluorescent light ratios and pollen grain sizes. Any competent technique or combinations of techniques may be used to automatically recognize or identify the collected pollen. Some examples of identification techniques include image processing, non-image optical properties such scattering and fluorescence, and others.

The operations of the system, such as the collection operations, can be logged and time-stamped. The time-stamping allows for a cross-referencing of the collected particles (and particle identifications) with the time of the allergic reaction. As one of skill in the art will recognize, the rate at which the conveyor assemblies advance their respective tapes can be synchronized or sequenced with the camera so that the tape portion having the collected particles is properly positioned with respect to the camera for imaging.

In a specific embodiment, the adhesive coated tape having the collected physical particles is archived and retained for possible future retrieval. The reel of tape can be removed from the particle collection device and sent to a laboratory for archiving and further analysis.

In a specific embodiment, a number of the intake vents or orifices and the incoming air can be as little as a single orifice or there can be multiple intake vents (e.g., two or more). In an embodiment having a single intake vent, it can be desirable (but not required) for the device to have a mechanism for aligning itself in the direction of incoming wind in order to sample as many particles flying through the air as possible or desired. For example, the mechanism may include wind vane to identify a direction of the wind. Once the direction of the wind is identified, the collection device may then automatically rotate or orient itself so that the intake orifice is aligned with the incoming wind.

In another specific embodiment, there are many or multiple intake orifices to cover the entire circumference (or a portion of the circumference) of the cylindrical device. In this specific embodiment, aligning the device in the direction of oncoming wind may not be necessary.

FIG. 16 shows a dimension D16 that indicates a diameter of the collection device and a dimension H16 that indicates a height of the collection device. In a specific embodiment, the diameter is about 100 mm and the height is about 150 mm. It should be appreciated, however, that these dimensions may vary greatly depending upon factors such as desired performance criteria, manufacturing cost targets, expected service life, expected operating environment, and many others. A cylindrical shape is preferred but not required. For example, in addition to providing an aesthetic appearance, a cylindrical monitor with a vertical axis of rotation can rotate to sample pollen from different orientations without risk of mechanical interference. Any shape or form factor may be suitable as long as there is an intake orifice in any orientation where the particles can be separated and analyzed as described by the mechanisms and techniques herein.

The air intake orifices or duct can be made to vibrate and oscillate to various frequencies such that particles that may have attached to the orifice or duct surface can separate from the surface and the air intake pull force will pull these in towards the surface of the non-stick tape.

In a specific embodiment, the adhesive-coated tape is transparent. This allows the optical imaging elements to be placed on a backside of the tape (i.e., a side of the tape opposite a side having the collected particles) and still be able to capture an image of the particles for analysis. There can be many different configurations of the particle collection device to meet desired form factors, performance, and so forth. Thus, it should be appreciated that the mechanical schematics shown in FIGS. 16-19 are merely an example of one particular implementation of the collection device.

In other implementations, other similar and equivalent elements and functions may be used or substituted in place of what is shown. For example, roller 1639C of the first conveyor assembly is shown as being aligned along a centerline or longitudinal axis of the collection device. The roller, however, may be offset from the centerline or closer one side of the collection device than an opposite side of the collection device so long as the adhesive-coated tape of the second conveyor assembly is suitably located to collect the particles which fall from the non-stick tape of the first conveyor assembly.

As another example, a vibration mechanism has been described as a technique to transfer particles from the non-stick tape of the first conveyor assembly to the adhesive-coated tape of the second conveyor assembly. In another specific embodiment, however, a vacuum, brush, or both may instead or additionally be used to transfer the particles. As another example, the optical imaging elements (e.g., camera) may be configured to capture images of the particles while the particles remain on the non-stick tape.

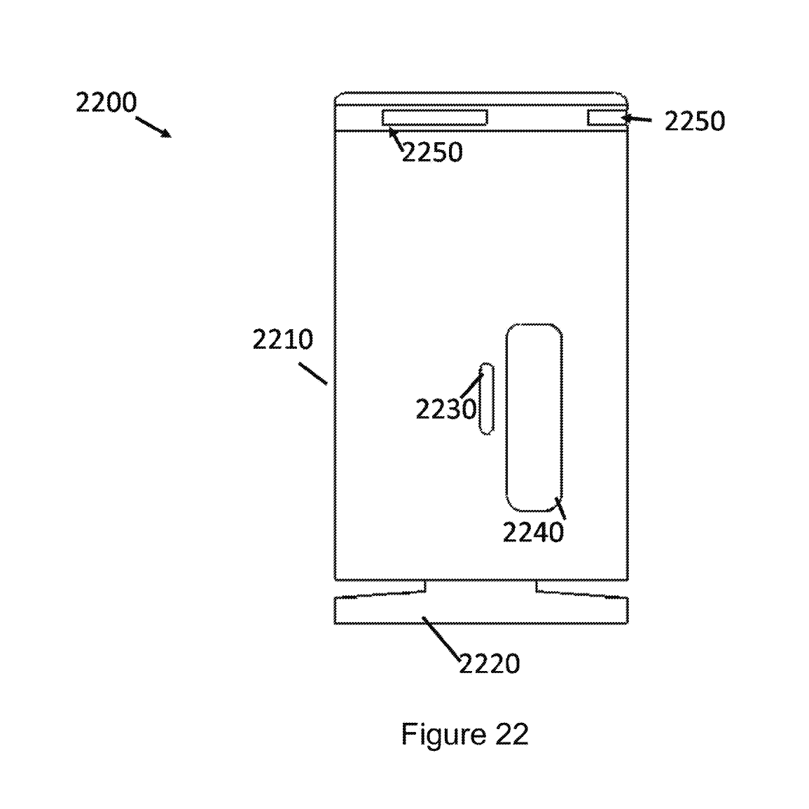

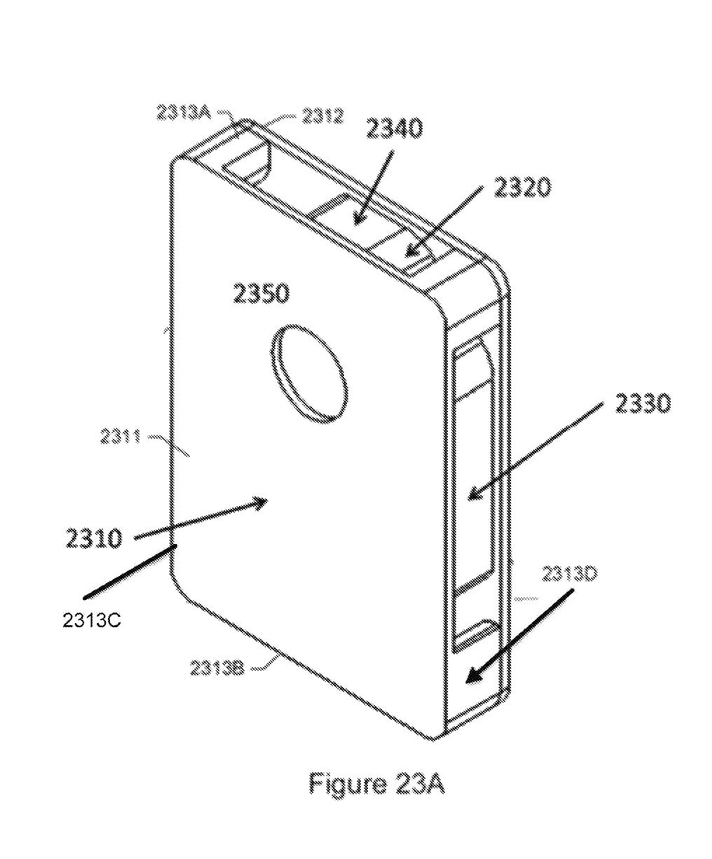

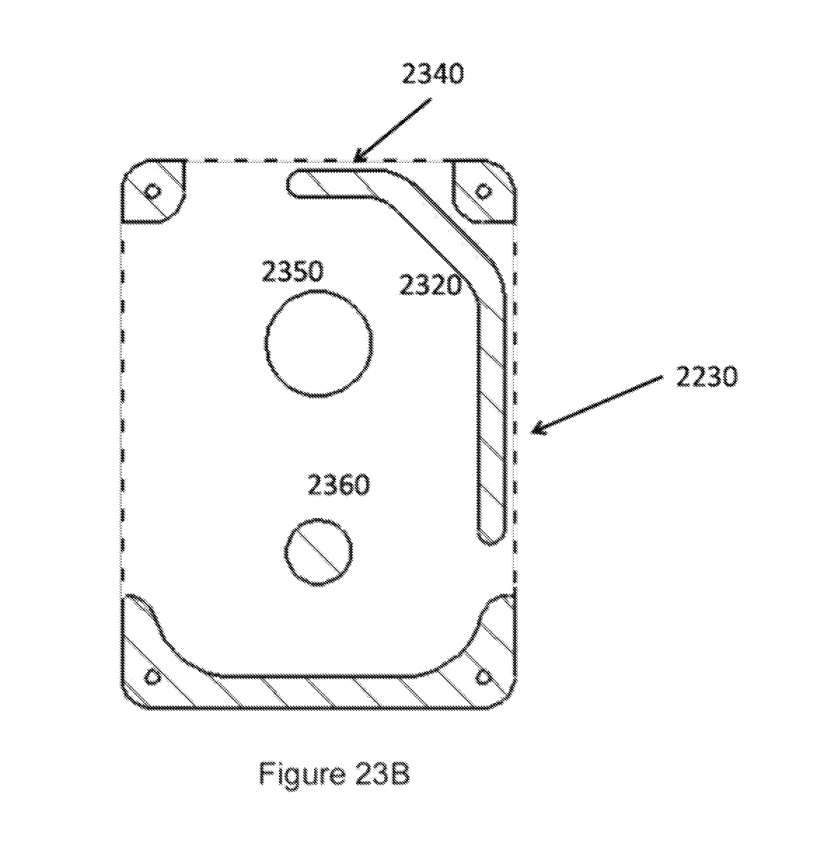

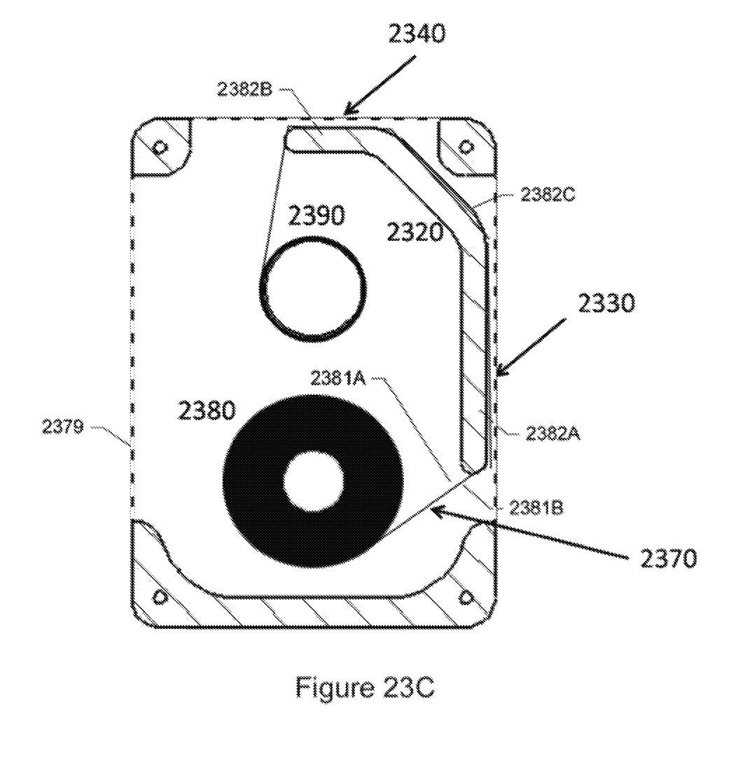

FIGS. 22 through 27A illustrate an alternate particle collection device 2200 according to another specific embodiment. FIG. 22 shows an exterior view including a cylindrical housing 2210 that contains most of the device components as well as a base 2220. Cylindrical housing 2210 contains an air-intake slot 2230 that may be a few centimeters in length and a width that varies from 3 mm to 1 mm in funnel-like fashion as it penetrates the thickness of the cylindrical housing 2210. The cylindrical housing 2210 also contains a particle-media-cartridge door 2240 that may be opened in order to insert or remove particle media cartridges such as shown in FIG. 23 and discussed below. The air-intake slot is adjacent or next to the cartridge door. A shape of the cartridge door includes a rectangle. The cartridge door is oriented vertically with respect to a central axis passing through the particle collection device.

The cylindrical housing 2210 and its contents may rotate about its cylindrical axis with respect to the base in order to orient the air-intake slot 2230 in a desired direction. In some cases, it may be desired to systematically vary the orientation of the air-intake slot 2230 in order to average over all directions. Alternatively, the particle collection device 2200 may orient itself so that the air-intake slot 2230 faces upwind to any breeze or other flow of ambient air. In this latter case, it is advantageous for the particle collection device 2200 to include wind or airflow sensors. Visible in FIG. 22 are two of four wind-detector recesses 2250 in which may be mounted airflow sensors in such a way that they are both exposed to ambient airflow and mechanically protected from accidental impact or contact. Wind detectors of many types, including hot-wire airflow detectors, may be placed in the wind-detector recesses 2250.

The generally cylindrical elongated shape of the housing helps to reduce interference with other external objects (e.g., furniture) when the collection device rotates to sample pollen from different directions. In this specific embodiment, a cross-sectional shape of the housing includes a circle. In other specific embodiments, a cross-sectional shape of the housing may include a square, rectangle, oval, triangle, or any other shape.

FIGS. 23A, 23B and 23C illustrate particle media cartridge that may be loaded or removed from the particle collection device 2200 via the particle-media-cartridge door 2240. The cartridge includes a media for capturing particles as well as a cartridge body 2310. In this specific embodiment the media is adhesive coated tape, however, in other embodiments a different media may be used such as adhesive coated slides. The cartridge body 2310 includes a tape guide structure 2320 that includes portions in an air-intake zone 2330 and a particle inspection zone 2340. The cartridge body 2310 includes a gear-shaft hole 2350 that will be discussed further below.

FIGS. 23B and 23C show a cross-section of the cartridge body with and without the media. The cross-section is for a plane parallel to, in the middle of, planes corresponding to front panel 2311 and back panel 2312. The dashed lines in FIGS. 23B and 23C represent portions of the plan-view edges of front panel 2311 and back panel 2312. Referring now to FIG. 23C, a supply reel 2380 of adhesive coated tape 2370 is mounted on supply-reel post 2360 (FIG. 23B). In the air-intake zone 2330, the tape guide structure 2320 both fixes the location of the adhesive coated tape 2370 where it collects particles in the face of air pressure from air entering the cylindrical housing 2210 via the air-intake slot 2230. The adhesive coated tape 2370 then passes the particle inspection zone 2340 and is finally collected at the uptake reel 2390. Optionally, after use within the particle collection device 2200, the particle-media cartridge may be removed from device 2200 and sent to a laboratory where particles captured by media can be further studied optically or with bio-assays.

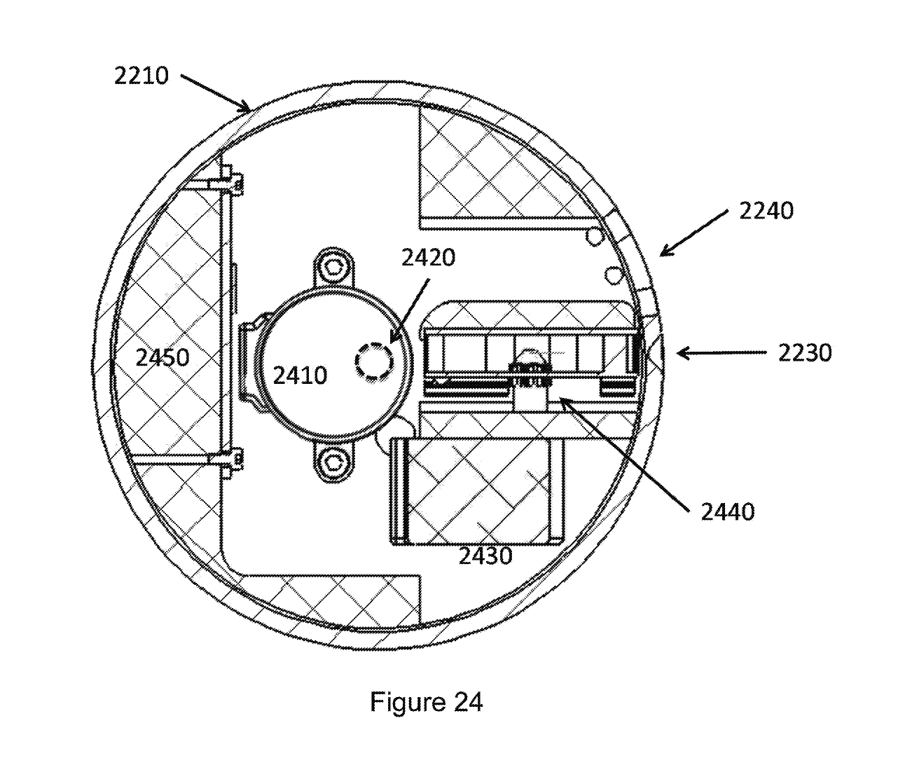

FIG. 24 shows a plan-view of selected items of the particle collection device 2200. The device includes two electric motors. Orientation motor 2410 rotates the cylindrical housing 2210 and its contents about its vertical axis and relative to the base 2220. While the orientation motor 2410 is not centered with respect to the axis of the cylindrical housing 2210, the orientation motor's gear shaft 2420 is centered. The intake-reel gear shaft 2440 of the cartridge-reel motor 2430 extends horizontally and controls the rotation of the uptake-reel 2390 (FIG. 23C) of the cartridge. The gear shaft hole 2350 (FIG. 23B) allows the intake-reel gear shaft 2440 to enter the particle-media cartridge body 2310 (FIG. 23A). Many of the contents contained within the cylindrical housing 2210, including motors 2410 and 2430, are mechanically supported by the internal mounting structure 2450. The internal mounting structure 2450 may be formed of a sculpted volume of plastic.

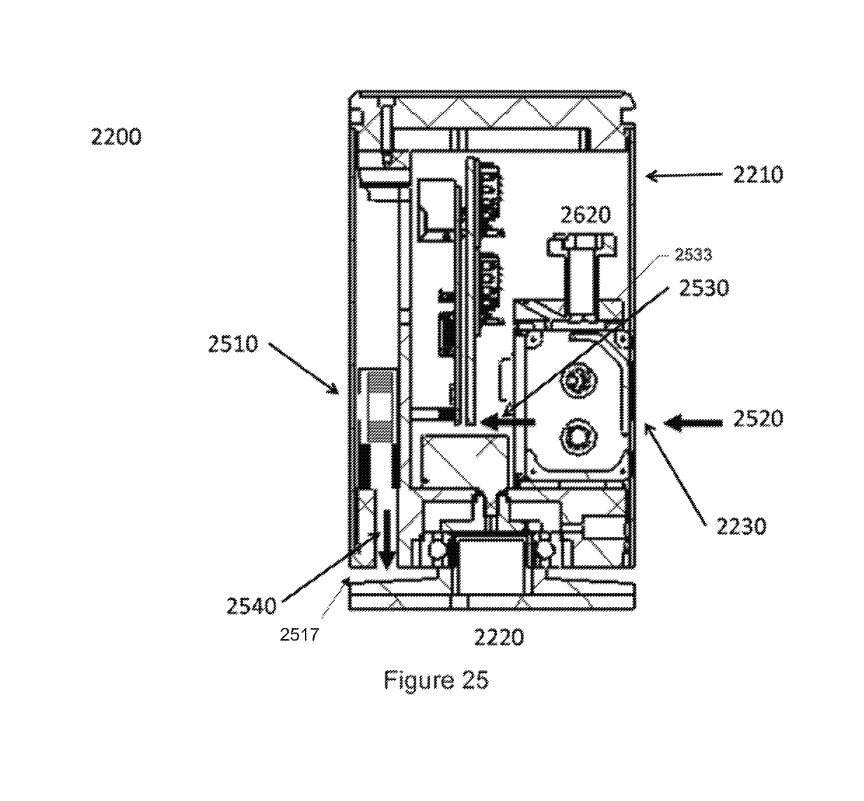

FIG. 25 illustrates how sampled ambient air flows through the alternate pollen collection device 2200. Sampled ambient air 2520 enters through the air-intake slot 2230 and immediately encounters the air-intake zone 2330 (FIG. 23C) of the particle-media cartridge. Here the adhesive-coated tape 2370 (FIG. 23C) captures many of the particles within the sampled ambient air 2520. Device-interior air 2530 then exits out the back side of the particle-media cartridge body 2310; for this purpose and as seen in FIG. 23B, the back side of the cartridge is open rather than closed. Finally exhaust air 2540 leaves the device. This airflow is driven by blower 2510 which pushes out exhaust air 2540 and sucks in sampled ambient air 2520. The blower is opposite the air intake slot and above the exhaust. A gap 2517 between a bottom of the housing and a top of the base allows the exhaust air to escape.

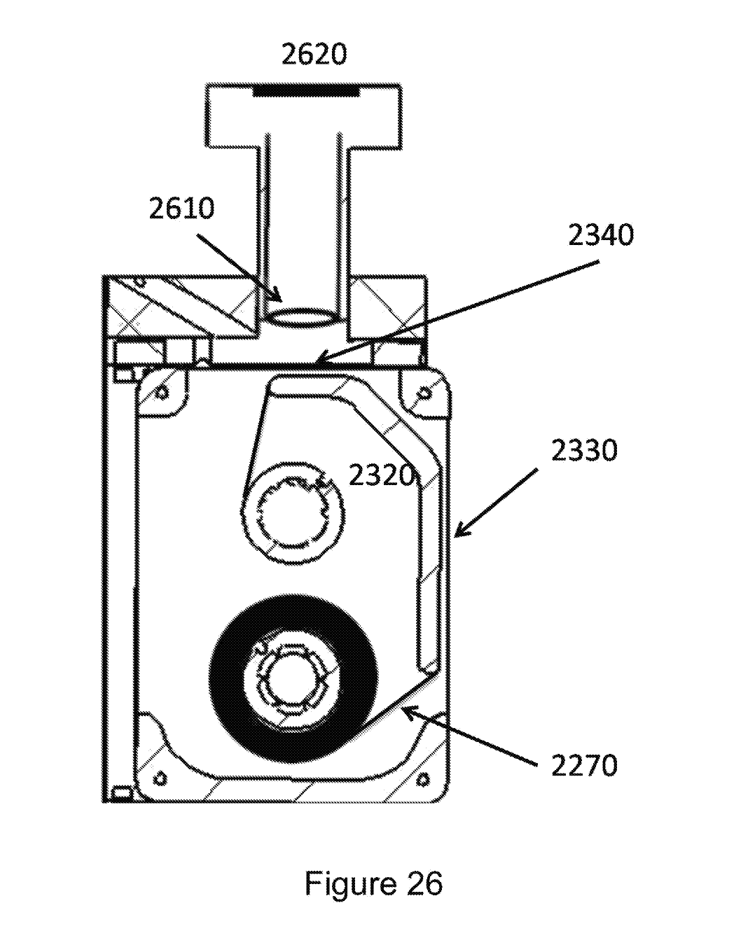

FIG. 26 illustrates a loaded particle-media cartridge along with an optical system for particle inspection. A lens assembly 2610 images the particle inspection zone 2340 on a camera sensor 2620. Optionally, the lens assembly 2610 may provide an electrically controlled focal length. Optionally, the camera sensor 2620 may capture RGB (red-green-blue) color images. Camera sensor 2620 may also be a light-field camera sensor. Visible light, UV light sources (not shown), or both illuminate the particle inspection zone 2340 so that camera sensor 2620 may image scattered light, fluorescent light, or both.

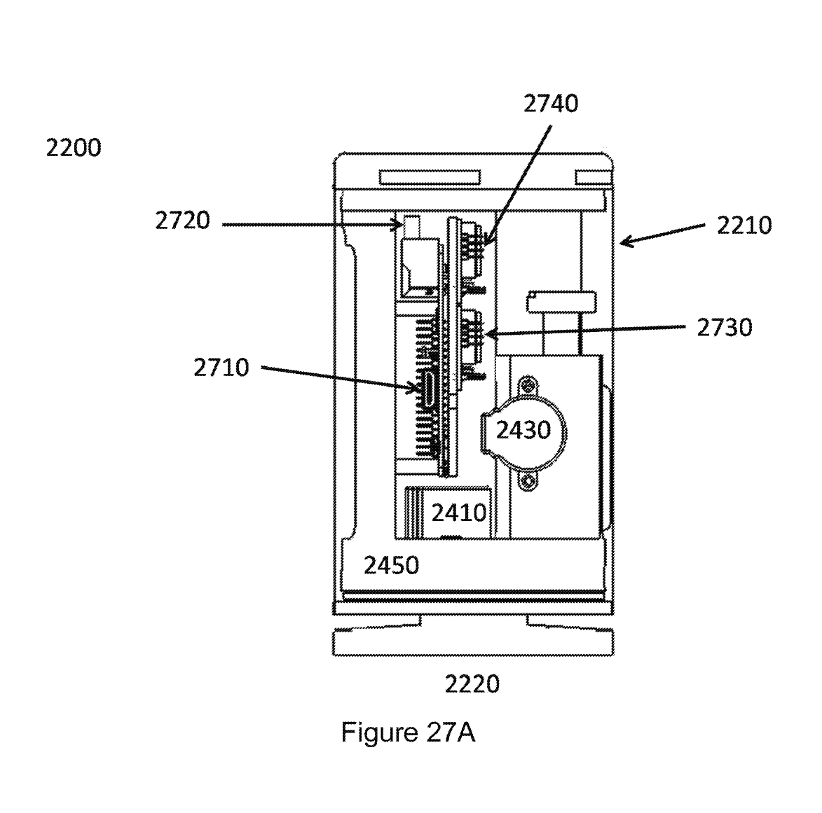

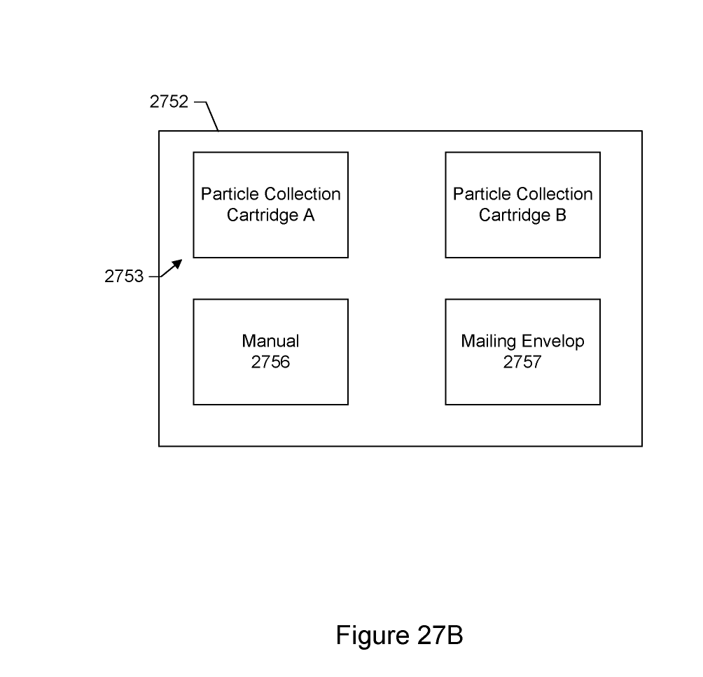

FIG. 27A shows a vertical cross section of particle collection device 2200 including three electronic boards. Motherboard 2710 contains many electronic components including a microprocessor (e.g., Raspberry Pi) and a wifi antenna 2720. Alternatively Bluetooth or any other wireless protocol may be used. For effective wireless communication, it is preferable that cylindrical housing 2210 be constructed from a non-conductive material such as plastic rather than a metal. Also shown in FIG. 27A are orientation-motor circuit board 2730 and cartridge reel motor circuit board 2740. Additional circuit boards (not shown) may be included. Also not shown in FIG. 27A for purposes of clarity are numerous wires interconnecting various components such as wires between the motors and their corresponding circuit boards.

In both the pollen collection device of FIGS. 16-19 and the alternate pollen collection device of FIGS. 22-27A, the adhesive coated tape (or other particle collection media such as adhesive coated glass slides) may be removed from the particle collection device and fresh media inserted into the particle collection device. Removed media containing captured particles may be subjected to laboratory inspection and testing, archived for possible future laboratory inspection and testing, or both.

Referring back now to FIGS. 23A-C, in a specific embodiment, a user-removable or replaceable particle media cartridge is provided. The cartridge includes a front panel 2311 (FIG. 23A), a back panel 2312, opposite the front panel. Side panels including a top side panel 2313A, a bottom side panel 2313B, a left side panel 2313C, and a right side panel 2313D extend between the front and back panels. The top and bottom side panels are opposite to each other. The left and right side panels are opposite to each other. The top and bottom side panels are orthogonal to the right and left side panels.

The right side panel includes a first opening that may be referred to as the air intake zone. The top side panel includes a second opening that may be referred to as the particle inspection zone. The left side panel includes a third opening that may be referred to as an exhaust port 2379 (FIG. 23C). The first opening, second opening, and third opening of the cartridge may extend over most of a length of their respective sides. This allows, for example, large air-intake and particle inspection regions (first and second cartridge openings), and a large air exhaust region (third cartridge opening).

Inside the cartridge is supply reel 2380 (FIG. 23C), the uptake reel, and the tape guide structure. The supply reel includes the roll of tape. The tape includes an inside surface 2381A and an outside surface 2381B, opposite the inside surface. The tape is wound so that inside surface faces towards a center of the roll, and the outside surface faces away from the center of the roll. The outside surface of the tape includes an adhesive. The inside surface of the tape may not include the adhesive and preferably moves with minimal or little friction against tape guide 2320.

The tape guide structure is sandwiched between the first and second panels of the cartridge. The structure includes a first segment 2382A, a second segment 2382B, orthogonal to the first segment, and a third segment 2382C extending between ends of the first and second segment. The first segment extends in a direction parallel to the right side panel and includes a surface that faces the first opening (e.g., air intake zone) of the cartridge. The second segment extends in a direction parallel to the top side surface and includes a surface that faces the second opening (e.g., particle inspection zone).

The tape extends from the supply reel, across the top surfaces of the first, second, and third segments of the tape guide structure, and terminates at the uptake reel. The tape is configured so that the inside surface contacts the top surfaces of the first, second, and third segments of the tape guide structure while the outside surface of the tape, which includes the adhesive, is exposed at the air intake and particle inspection zones. Thus, particles entering the air intake zone can be trapped by the adhesive and then inspected at the particle inspection zone. The air can pass from the air intake zone and out the exhaust port of the cartridge. The inside surface of the tape may be smooth or without the adhesive so that the tape can glide across the tape guide structure.

In a specific embodiment, a set of particles collected on a portion of the tape that is exposed within the air intake zone is tagged with metadata such as a time and date of the collection, geographical location of the collection, other metadata, or combinations of these. The metadata, such as the time and date of the collection, can be used to cross-reference, correlate, group, or assign the collected particles to the priming, pre-aggravation, and aggravation periods.

Referring back now to FIG. 25, camera sensor 2620 is mounted on a platform 2533. The platform is above a docking structure that receives the collection cartridge. The docking structure may be referred to as a deck or well. The camera sensor is positioned within the particle collection device to be above or over the second opening or particle inspection zone of the cartridge. The camera is closer to a top of the particle collection device than the cartridge. Positioning the camera above the particle inspection zone helps to reduce the probability of particles falling onto the camera lens and obscuring the images. For example, in some cases, the bond between the adhesive coated tape and collected airborne particle may be weak, the adhesive coated tape may include a large collection or mound of particles and particles at the top of the mound may not be secured to the adhesive coated tape, and so forth. In a specific embodiment, the components of the particle collection device are positioned or arranged to provide a compact and space-efficient form factor. This facilitates placement of the collection device in, for example, a user's home or office. The collection cartridge and camera may be aligned such that a line passing through the supply and uptake reels passes through the particle inspection zone and lens of the camera.

Referring back now to FIGS. 22, 25, and 26, air intake slot 2230 is opposite the blower and is configured to direct a flow path of ambient air created by the blower towards or over the first opening of the cartridge or air intake zone. For example, there can be channel, duct, conduit, tube, or passageway that directs the flow path of the air from the air intake zone. Particles such as pollen in the air are trapped by the adhesive on the tape. Preferably, the airflow in the air intake zone is turbulent in order to maximize or improve the chances that particles in the sampled air will be separated from the air and adhered to the capturing medium. The air intake zone is the upstream end of the flow path of the air. Any channel or duct can be downstream of that. When desired, cartridge reel motor 2430 (FIG. 24) advances the tape containing the trapped particles to the second opening of the cartridge or inspection zone. The camera can then capture images of the pollen trapped by the adhesive tape.

FIG. 27B shows an example of a kit 2752 including a set of replaceable particle collection cartridges 2753. In the example shown in FIG. 27B, the kit includes a box and a tray inside the box. The tray holds particle collection cartridge A, particle collection cartridge B, an instruction manual 2756, and a mailing envelope 2757.

The kit may or may not include the particle collection device. The instruction manual provides instructions for inserting a cartridge into the particle collection device, removing the cartridge from the particle collection device, and (if desired) mailing a used cartridge to a laboratory for further analysis of the particles that may have been trapped. The mailing envelop can be a pre-paid and pre-addressed mailing envelop that the user can use to mail the cartridge. In an embodiment, the user purchases a particle collection device and can separately purchase additional blank collection cartridges as desired. In the example shown in FIG. 27B, two collection cartridges are shown. It should be appreciated, however, that a kit may include any number of cartridges such as one, two, three, four, five, or more than five cartridges.

A specific application of the system is the monitoring of allergens. Aspects and principles of the system, however, may be applied to other fields including the study of pollen, i.e., palynology. The collection and analysis of pollen plays an important role in a number of scientific and applied fields including agriculture and ecology including climate change effects on seasonal timing and geographical distribution of airborne pollens. Previous approaches to capturing and analyzing airborne pollen often involved a great deal of manual work and expensive and bulky equipment. The system including the pollen collection devices described herein, however, provides an automated, compact, and cost-effective approach to collecting and analyzing pollen.

It should be appreciated that while some embodiments described above discuss allergic reactions to pollen, one of skill in the art will recognize that aspects and principles of the system may be applied to other airborne allergens.

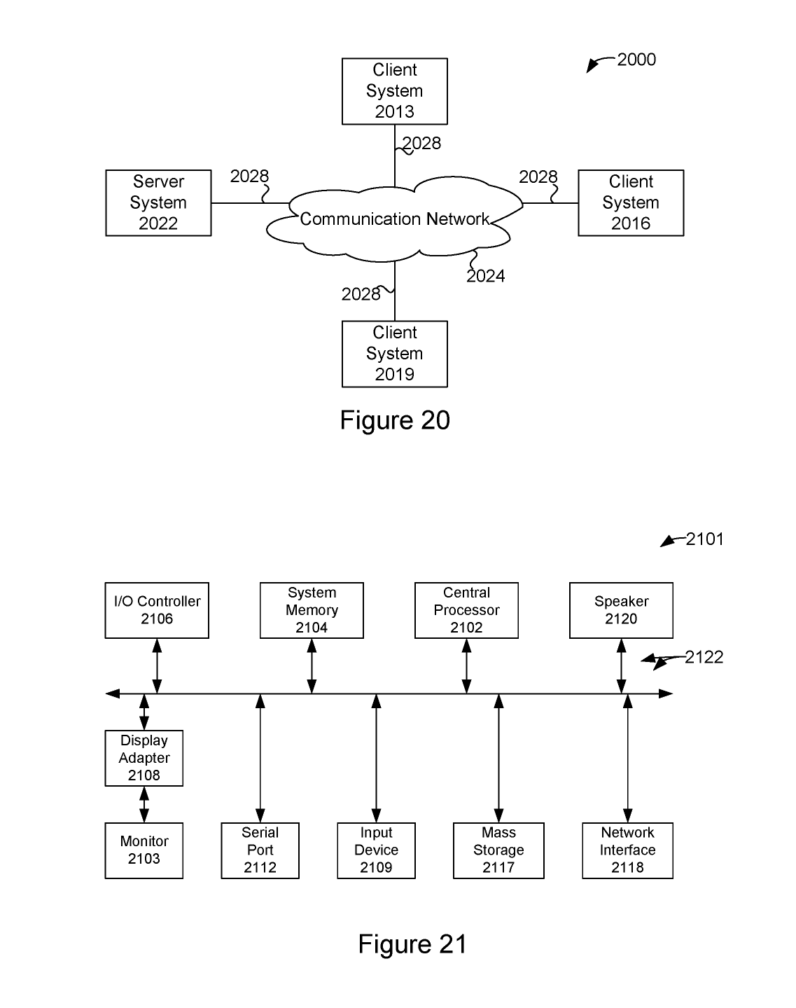

FIG. 20 is a simplified block diagram of a distributed computer network 2000 that may be used in a specific embodiment of the system. Computer network 2000 includes a number of client systems 2013, 2016, and 2019, and a server system 2022 coupled to a communication network 2024 via a plurality of communication links 2028. There may be any number of clients and servers in a system. Communication network 2024 provides a mechanism for allowing the various components of distributed network 2000 to communicate and exchange information with each other.

Communication network 2024 may itself be comprised of many interconnected computer systems and communication links. Communication links 2028 may be hardwire links, optical links, satellite or other wireless communications links, wave propagation links, or any other mechanisms for communication of information. Various communication protocols may be used to facilitate communication between the various systems shown in FIG. 20. These communication protocols may include TCP/IP, HTTP protocols, wireless application protocol (WAP), vendor-specific protocols, customized protocols, and others. While in one embodiment, communication network 2024 is the Internet, in other embodiments, communication network 2024 may be any suitable communication network including a local area network (LAN), a wide area network (WAN), a wireless network, an intranet, a private network, a public network, a switched network, and combinations of these, and the like.

Distributed computer network 2000 in FIG. 20 is merely illustrative of an embodiment and is not intended to limit the scope of the embodiment as recited in the claims. One of ordinary skill in the art would recognize other variations, modifications, and alternatives. For example, more than one server system 2022 may be connected to communication network 2024. As another example, a number of client systems 2013, 2016, and 2019 may be coupled to communication network 2024 via an access provider (not shown) or via some other server system.

Client systems 2013, 2016, and 2019 enable users to access and query information stored by server system 2022. In a specific embodiment, a “Web browser” application executing on a client system enables users to select, access, retrieve, or query information stored by server system 2022. Examples of web browsers include the Internet Explorer® browser program provided by Microsoft® Corporation, Chrome® browser provided by Google®, and the Firefox® browser provided by Mozilla® Foundation, and others. In another specific embodiment, an iOS App or an Android® App on a client tablet enables users to select, access, retrieve, or query information stored by server system 2022. Access to the system can be through a mobile application program or app that is separate from a browser.

A computer-implemented or computer-executable version of the system may be embodied using, stored on, or associated with computer-readable medium or non-transitory computer-readable medium. A computer-readable medium may include any medium that participates in providing instructions to one or more processors for execution. Such a medium may take many forms including, but not limited to, nonvolatile, volatile, and transmission media. Nonvolatile media includes, for example, flash memory, or optical or magnetic disks. Volatile media includes static or dynamic memory, such as cache memory or RAM. Transmission media includes coaxial cables, copper wire, fiber optic lines, and wires arranged in a bus. Transmission media can also take the form of electromagnetic, radio frequency, acoustic, or light waves, such as those generated during radio wave and infrared data communications.

For example, a binary, machine-executable version, of the software of the present system may be stored or reside in RAM or cache memory, or on a mass storage device. The source, executable code, or both of the software may also be stored or reside on a mass storage device (e.g., hard disk, magnetic disk, tape, or CD-ROM). As a further example, code may be transmitted via wires, radio waves, or through a network such as the Internet.

A client computer can be a smartphone, smartwatch, tablet computer, laptop, wearable device or computer (e.g., Google Glass), body-borne computer, or desktop.

FIG. 21 shows a system block diagram of computer system 2101. Computer system 2101 includes monitor 2103, input device (e.g., keyboard, microphone, or camera) 2109, and mass storage devices 2117. Computer system 2101 further includes subsystems such as central processor 2102, system memory 2104, input/output (I/O) controller 2106, display adapter 2108, serial or universal serial bus (USB) port 2112, network interface 2118, and speaker 2120. In an embodiment, a computer system includes additional or fewer subsystems. For example, a computer system could include more than one processor 2102 (i.e., a multiprocessor system) or a system may include a cache memory.

Arrows such as 2122 represent the system bus architecture of computer system 2101. However, these arrows are illustrative of any interconnection scheme serving to link the subsystems. For example, speaker 2120 could be connected to the other subsystems through a port or have an internal direct connection to central processor 2102. The processor may include multiple processors or a multicore processor, which may permit parallel processing of information. Computer system 2101 shown in FIG. 21 is but an example of a suitable computer system. Other configurations of subsystems suitable for use will be readily apparent to one of ordinary skill in the art.

Computer software products may be written in any of various suitable programming languages, such as C, C++, C#, Pascal, Fortran, Perl, Matlab® (from MathWorks), SAS, SPSS, JavaScript®, AJAX, Java®, SQL, and XQuery (a query language that is designed to process data from XML files or any data source that can be viewed as XML, HTML, or both). The computer software product may be an independent application with data input and data display modules. Alternatively, the computer software products may be classes that may be instantiated as distributed objects. The computer software products may also be component software such as Java Beans® (from Oracle Corporation) or Enterprise Java Beans® (EJB from Oracle Corporation). In a specific embodiment, a computer program product is provided that stores instructions such as computer code to program a computer to perform any of the processes or techniques described.

An operating system for the system may be iOS by Apple®, Inc., Android by Google®, one of the Microsoft Windows® family of operating systems (e.g., Windows NT®, Windows 2000®, Windows XP®, Windows XP® x64 Edition, Windows Vista®, Windows 7®, Windows CE®, Windows Mobile®, Windows 8), Linux, HP-UX, UNIX, Sun OS®, Solaris®, Mac OS X®, Alpha OS®, AIX, IRIX32, or IRIX64. Other operating systems may be used. Microsoft Windows® is a trademark of Microsoft® Corporation.

Furthermore, the computer may be connected to a network and may interface to other computers using this network. The network may be an intranet, internet, or the Internet, among others. The network may be a wired network (e.g., using copper), telephone network, packet network, an optical network (e.g., using optical fiber), or a wireless network, or any combination of these. For example, data and other information may be passed between the computer and components (or steps) of the system using a wireless network using a protocol such as Wi-Fi (IEEE standards 802.11, 802.11a, 802.11b, 802.11e, 802.11g, 802.11i, and 802.11n, just to name a few examples). For example, signals from a computer may be transferred, at least in part, wirelessly to components or other computers.

In an embodiment, with a Web browser executing on a computer workstation system, a user accesses a system on the World Wide Web (WWW) through a network such as the Internet. The Web browser is used to download web pages or other content in various formats including HTML, XML, text, PDF, and postscript, and may be used to upload information to other parts of the system. The Web browser may use uniform resource identifiers (URLs) to identify resources on the Web and hypertext transfer protocol (HTTP) in transferring files on the Web.

In a specific embodiment, there is a pollen (or other allergen or pathogen) collection system including a pollen sampler and an allergic reaction monitor in which pollen sampling is triggered when the monitor detects an allergic reaction on the part of the user. Control samples may also be collected when no allergic reaction is detected by the monitor. The pollen collection system may be capable of communicating to an information network. Information about samples collected may be communicated to the network. Information relevant to the interpretation of pollen samples may be received from the network.

The allergic reaction monitor may include a microphone and audio signal processing capability. The microphone of the allergic-reaction monitor can be a user wearable device. The allergic-reaction monitor can be a user wearable device capable of monitoring at least one physiological parameter of the user.

In a specific embodiment, the pollen sampler includes an adhesive surface upon which pollen grains are captured. The adhesive surface may be on tape from a reel or a removable glass slide.

The pollen collection system may further include a pollen detection system. The pollen detection system may include a camera capable of producing images of pollen grains. There can be image processing capable of identifying pollen types.

In a specific embodiment, non-image optical properties of pollen grains are measured. Non-image optical properties of pollen grains may include scattering properties. Non-image optical properties of pollen grains may include fluorescence.

The pollen sampler may be installed and integrated into an automobile. The pollen sampler may be powered by the automobile's electrical system. The pollen sampler can sample air that is external to the passenger compartment of the automobile.

In a specific embodiment, a method includes receiving from a sensor local to a user information indicating that the user has experienced a physiological event; analyzing the information to determine whether the physiological event should be classified as an allergic reaction; and if the physiological event should be classified as the allergic reaction, collecting particles currently present in an environment local to the user.

The method may include periodically collecting over a rolling time period particles in the environment local to the user; and correlating the currently collected particles with the particles collected over the rolling time period to identify an aggravating allergen, wherein the aggravating allergen is among the currently collected particles.

The method may include periodically collecting over a rolling time period particles in the environment local to the user; and correlating the currently collected particles with the particles collected over the rolling time period to identify a priming allergen, wherein the priming allergen is present among the particles collected over the rolling time period.

The sensor may include a microphone. The sensor may include an accelerometer.

Analyzing the information may include prompting the user to confirm whether the physiological event should be classified as the allergic reaction. The method may include prompting the user to simulate the allergic reaction; and generating an allergic reaction signature based on the simulated allergic reaction, wherein the analyzing the information comprises comparing the information indicating that the user has experienced the physiological event against the allergic reaction signature.

The particles may include at least one of pollen or mold spore. The particles may include airborne particles.

In another specific embodiment, a method includes periodically collecting over a rolling time period candidate priming pollens in an environment local to a user; receiving from a sensor local to the user information indicating that the user has experienced a physiological event; analyzing the information to determine whether the physiological event should be classified as an allergic reaction; based on the analysis, determining that the physiological event should be classified as the allergic reaction; upon the determination, designating pollens collected before a time of the allergic reaction as being candidate aggravating pollens; identifying the candidate aggravating pollens and the candidate priming pollens; scanning a table comprising a listing of pollens, and a listing of priming pollens corresponding to the listing of pollens to find a specific pollen among the listing of pollens that is present in the candidate aggravating pollens, and a specific priming pollen among the listing of priming pollens, corresponding to the specific pollen, that is present in the candidate priming pollens; and generating a notification comprising an identification of the specific pollen, and an identification of the specific priming pollen that corresponds to the specific pollen.

The method may include adjusting the rolling time period from a first duration to a second duration, different from the first duration.

In an embodiment, the sensor is a first sensor, the first sensor comprises a microphone, the information is first information, and the method includes receiving from a second sensor second information indicating that the user has experienced the physiological event, wherein the second sensor comprises an accelerometer, and wherein the analyzing the information comprises analyzing both the first and second information to determine whether the physiological event should be classified as the allergic reaction.

The method may include upon the receiving from a sensor local to the user information indicating that the user has experienced a physiological event, prompting the user to verify whether the physiological event should be classified as the allergic reaction.

The physiological event may include at least one of a cough or a sneeze. The information may include audio information. The information may include motion information.

In another specific embodiment, a method includes collecting over a rolling time period airborne particles in an environment local to a user; receiving from a sensor associated with the user information indicating that the user has experienced a physiological event; analyzing the information to determine whether the physiological event should be classified as an allergic reaction; if the physiological event should be classified as an allergic reaction, correlating first particles collected during a first portion of the rolling time period with second particles collected during a third portion of the rolling time period to identify an aggravating allergen among the first particles, and a priming allergen, corresponding to the aggravating allergen, among the second particles, wherein a duration of the first portion of the rolling time period is less than a duration of the third portion of the rolling time period, and wherein the first portion of the rolling time period is closer to a time of the physiological event than the third portion of the rolling time period.

The method may include storing the airborne particles on a removable collection cartridge. The removable collection cartridge may include a supply reel coupled inside the removable collection cartridge, and a tape comprising a first side, and a second side opposite the first side, wherein the second side comprises an adhesive, and the first side does not comprise the adhesive, wherein the tape is wound about the supply reel, and arranged so that the second side comprising the adhesive faces away from a center of the supply reel, and the first side not comprising the adhesive faces towards the center of the supply reel; and wherein the airborne particles are trapped by the adhesive on the second side.

In a specific embodiment, there is a system for analyzing pollen comprising: a particle collection device; and a collection cartridge that is removable from the particle collection device, wherein the collection cartridge comprises: a first panel; a second panel, opposite the first panel; first, second, and third sides extending between the first and second panels, the first side being orthogonal to the second side, and the third side being opposite the first side; a first opening on the first side that defines an air intake region; a second opening on the second side that defines an inspection region; a third opening on the third side that defines an exhaust region; supply and uptake reels coupled between the first and second panels; a tape wound about the supply and uptake reels, and comprising a first side, and a second side, opposite the first side, the second side comprising an adhesive, and the first side not comprising the adhesive; and a tape guide structure coupled between the first and second panels, the tape guide structure comprising an air intake segment within the air intake region, and an inspection segment within the inspection region, wherein the tape is arranged to extend from the supply reel, across the air intake and inspection segments, and terminate at the uptake reel, and positioned so that the second side comprising the adhesive is exposed at the air intake and inspection regions.

The particle collection device comprises: a base; a cylindrical elongated body, rotatably coupled to the base, and comprising: an air intake opening that passes from an outside of the body to an inside of the body; an air outtake opening between a bottom of the body and the base; a blower opposite the air intake opening; a collection cartridge slot adjacent to the air intake opening that receives the collection cartridge; a first motor coupled to rotate the body about the base; a second motor coupled to advance the tape of the collection cartridge across the air intake and inspection regions; and a camera coupled above the inspection region, wherein the blower generates a flow path of air, the flow path of air being through the air intake opening of the body, through the air intake region of the cartridge, out the exhaust region of the cartridge, and out the air outtake opening of the body.

It should be emphasized that pollen is often given in the above described embodiments, it is understood that similar methods may be applied to other allergenic particles such as mold spores, animal (e.g., tiny flecks of skin shed by cats, dogs, and birds), or any other allergenic particles that may be present in the user's personal or local environment.

In the description above and throughout, numerous specific details are set forth in order to provide a thorough understanding of an embodiment of this disclosure. It will be evident, however, to one of ordinary skill in the art, that an embodiment may be practiced without these specific details. In other instances, well-known structures and devices are shown in block diagram form to facilitate explanation. The description of the preferred embodiments is not intended to limit the scope of the claims appended hereto. Further, in the methods disclosed herein, various steps are disclosed illustrating some of the functions of an embodiment. These steps are merely examples, and are not meant to be limiting in any way. Other steps and functions may be contemplated without departing from this disclosure or the scope of an embodiment. Other embodiments include systems and non-volatile media products that execute, embody or store processes that implement the methods described above.

What is claimed is:

1. A method comprising:
receiving from a sensor local to a user information indicating that the user has experienced a physiological event;
analyzing the information to determine whether the physiological event should be classified as an allergic reaction; and
if the physiological event should be classified as the allergic reaction, collecting particles currently present in an environment local to the user.

2. The method of claim 1 comprising:
periodically collecting over a rolling time period particles in the environment local to the user; and
correlating the currently collected particles with the particles collected over the rolling time period to identify an aggravating allergen, wherein the aggravating allergen is among the currently collected particles.

3. The method of claim 2 wherein the rolling time period ranges from about 10 minutes to about several hours.

4. The method of claim 1 comprising:
periodically collecting over a rolling time period particles in the environment local to the user; and
correlating the currently collected particles with the particles collected over the rolling time period to identify a priming allergen, wherein the priming allergen is present among the particles collected over the rolling time period.

5. The method of claim 1 wherein the sensor comprises a microphone.

6. The method of claim 1 wherein the sensor comprises an accelerometer.

7. The method of claim 1 wherein the analyzing the information comprises:
prompting the user to confirm whether the physiological event should be classified as the allergic reaction.

8. The method of claim 1 comprising:
prompting the user to simulate the allergic reaction; and
generating an allergic reaction signature based on the simulated allergic reaction, wherein the analyzing the information comprises comparing the information indicating that the user has experienced the physiological event against the allergic reaction signature.

9. The method of claim 1 wherein the particles comprise at least one of pollen or mold spore.

10. The method of claim 1 wherein the particles comprise airborne particles.

11. A method comprising:
periodically collecting over a rolling time period candidate priming pollens in an environment local to a user;
receiving from a sensor local to the user information indicating that the user has experienced a physiological event;
analyzing the information to determine whether the physiological event should be classified as an allergic reaction;
based on the analysis, determining that the physiological event should be classified as the allergic reaction;
upon the determination, designating pollens collected before a time of the allergic reaction as being candidate aggravating pollens;
identifying the candidate aggravating pollens and the candidate priming pollens;
scanning a table comprising a listing of pollens, and a listing of priming pollens corresponding to the listing of pollens to find a specific pollen among the listing of pollens that is present in the candidate aggravating pollens, and a specific priming pollen among the listing of priming pollens, corresponding to the specific pollen, that is present in the candidate priming pollens; and
generating a notification comprising an identification of the specific pollen, and an identification of the specific priming pollen that corresponds to the specific pollen.

12. The method of claim 11 comprising:
adjusting the rolling time period from a first duration to a second duration, different from the first duration.

13. The method of claim 11 wherein the sensor is a first sensor, the first sensor comprises a microphone, the information is first information, and the method comprises:
receiving from a second sensor second information indicating that the user has experienced the physiological event, wherein the second sensor comprises an accelerometer, and
wherein the analyzing the information comprises analyzing both the first and second information to determine whether the physiological event should be classified as the allergic reaction.

14. The method of claim 11 comprising:
upon the receiving from a sensor local to the user information indicating that the user has experienced a physiological event, prompting the user to verify whether the physiological event should be classified as the allergic reaction.

15. The method of claim 11 wherein the physiological event comprises at least one of a cough or a sneeze.

16. The method of claim 11 wherein the information comprises audio information.

17. The method of claim 11 wherein the information comprises motion information.

18. A method comprising:
collecting over a rolling time period airborne particles in an environment local to a user;
receiving from a sensor associated with the user information indicating that the user has experienced a physiological event;
analyzing the information to determine whether the physiological event should be classified as an allergic reaction;
if the physiological event should be classified as an allergic reaction, correlating first particles collected during a first portion of the rolling time period with second particles collected during a third portion of the rolling time period to identify an aggravating allergen among the first particles, and a priming allergen, corresponding to the aggravating allergen, among the second particles,
wherein a duration of the first portion of the rolling time period is less than a duration of the third portion of the rolling time period, and
wherein the first portion of the rolling time period is closer to a time of the physiological event than the third portion of the rolling time period.

19. The method of claim 18 wherein the collecting comprises:
storing the airborne particles on a removable collection cartridge.

20. The method of claim 19 wherein the removable collection cartridge comprises a supply reel coupled inside the removable collection cartridge, and a tape comprising a first side, and a second side opposite the first side,
wherein the second side comprises an adhesive, and the first side does not comprise the adhesive,
wherein the tape is wound about the supply reel, and arranged so that the second side comprising the adhesive faces away from a center of the supply reel, and the first side not comprising the adhesive faces towards the center of the supply reel; and
wherein the airborne particles are trapped by the adhesive on the second side.
